(19) United States
(12) Patent Application Publication
(10) Pub. No.: US 2024/0399152 A1
(43) Pub. Date: Dec. 5, 2024

(54) TREATMENT OF MOTOR IMPAIRMENT AND/OR PROPRIOCEPTION IMPAIRMENT DUE TO NEUROLOGICAL DISORDER OR INJURY

(71) Applicant: University of Pittsburgh - Of the Commonwealth System of Higher Education, Pittsburgh, PA (US)

(72) Inventors: Marc Philip Powell, Pittsburgh, PA (US); Marco Capogrosso, Pittsburgh, PA (US); Douglas J. Weber, Pittsburgh, PA (US); Nikhil Verma, Pittsburgh, PA (US); Peter C. Gerszten, Pittsburgh, PA (US); Elvira Pirondini, Pittsburgh, PA (US); Erynn Sorensen, Pittsburgh, PA (US); Erick Fabrizio Carranza Urquizo, Pittsburgh, PA (US); Lee Fisher, Pittsburgh, PA (US)

(73) Assignees: University of Pittsburgh - Of the Commonwealth System of Higher Education, Pittsburgh, PA (US); CARNEGIE MELLON UNIVERSITY, Pittsburgh, PA (US)

(21) Appl. No.: 18/690,998

(22) PCT Filed: Sep. 9, 2022
(86) PCT No.: PCT/US2022/043128

§ 371 Date: Mar. 11, 2024

Related U.S. Application Data
(60) Provisional application No. 63/242,262, filed on Sep. 9, 2021.

(51) Int. Cl. A61N 1/36; A61N 1/05

(52) U.S. Cl. CPC A61N 1/36103; A61N 1/0553; A61N 1/36003; A61N 1/36067; A61N 1/36121; A61N 1/36171; A61N 1/36175; A61N 1/36185; A61N 1/36034

(57) ABSTRACT

Disclosed herein are methods for treating impairment of a limb in a subject. Particular methods comprise applying a therapeutically effective amount of an electrical stimulus to dorsal roots, dorsal rootlets, or dorsal root ganglia, of sensory neurons innervating the limb of the subject, wherein the impairment is a motor impairment and/or a proprioception impairment due to neurological disorder or injury, the electrical stimulus is applied with one or more electrodes controlled by a neurostimulator, and the one or more electrodes are implanted in the epidural space at the dorsolateral aspect of the spinal cord and proximate to the dorsal roots or dorsal rootlets of the sensory neurons innervating the limb of the subject, or the one or more electrodes are implanted proximate to dorsal root ganglia of sensory neurons innervating the limb of the subject.

### CROSS-REFERENCE TO RELATED APPLICATION

This application claims priority to U.S. Provisional Application No. 63/242,262, filed Sep. 9, 2021, which is incorporated by reference in its entirety.

### FIELD

The present disclosure relates to a method of treating motor impairment and/or proprioception impairment in a limb of a subject by stimulating the dorsal rootlets, or dorsolateral spinal cord or dorsal root ganglion adjacent to the dorsal rootlets, that innervate the affected limb in the subject.

### BACKGROUND

Implantable neurostimulation systems have proven therapeutic in a wide variety of diseases and disorders. For example, Spinal Cord Stimulation (SCS) techniques that directly stimulate the spinal cord tissue of the patient are approved for the treatment of chronic neuropathic pain syndromes, and the application of spinal cord stimulation has expanded to include additional applications, such as angina pectoralis, peripheral vascular disease, and incontinence. An implantable SCS system typically includes one or more electrode-carrying stimulation leads, which are implanted at a stimulation site in proximity to the spinal cord tissue of the patient, and a neurostimulator coupled either directly to the stimulation leads or indirectly to the stimulation leads via a lead connector to stimulate or activate a volume of the spinal cord tissue.

Many patients have disability from loss of motor and/or proprioception function due to neurological disorder or injury, such as in the case of stroke. Stroke is one of the largest causes of permanent disability in the United States. More than 800,000 people are affected by stroke every year and more than 50% of stroke survivors are affected by motor deficits. $46 billion was spent in a single year (2014/2015) for direct and indirect costs of care of this population. Permanent motor deficits, especially of the arm and hand, affect daily quality of life, social interactions, professional life, and mental health of both stroke survivors as well as their immediate family members.

Unfortunately, there are limited options to treat motor and proprioception impairment due to neurological disorder or injury, such as stroke, and outcomes are highly variable. Patients with moderate to severe paresis do not respond satisfactorily to physical therapy; some do not respond at all. Unfortunately, hundreds of thousands of patients are left with significant permanent motor impairment following stroke, particularly impairment of the arm and hand.

Thus, a need exists for new and improved treatments for motor and/or proprioception impairment, particularly in the case of such impairment induced by stroke.

### SUMMARY

Provided herein are methods for treating a motor impairment and/or a proprioception impairment of a limb in a subject. The impairment is a due to a neurological disorder or injury. The methods comprise applying, with one or more electrodes controlled by a neurostimulator, a therapeutically effective amount of an electrical stimulus to sensory neurons innervating the limb. In some implementations, the therapeutically effective amount of an electrical stimulus is applied to dorsal roots, dorsal rootlets, or dorsal root ganglia, of the sensory neurons, and the one or more electrodes are implanted in the epidural space at the dorsolateral aspect of the spinal cord and proximate to the dorsal roots or dorsal rootlets of the sensory neurons innervating the limb of the subject, or the one or more electrodes are implanted proximate to dorsal root ganglia of sensory neurons innervating the limb of the subject. Application of the electrical stimulus reduces the motor impairment and/or proprioception impairment of the limb of the subject.

In several implementations, the impairment is due to stroke. In several implementations, the limb is an arm and, for example, the one or more electrodes are contained within one or more electrode arrays implanted at the dorsolateral aspect of the spinal cord and spanning the C3-T1 nerve roots.

In several implementations, the impairment is a motor impairment, for example, an impairment of voluntary movement of the limb. In some implementations, the motor impairment comprises reduced muscle control, reduced muscle function, reduced muscle strength, partial paralysis, uncontrollable muscle tone, reduced dexterity, spasticity, contractures, and/or abnormal flexor synergy or contractures.

In some implementations, the electrical stimulus is applied at or below a motor threshold such that the electrical stimulus does not directly elicit movement of the impaired limb, and above a perceptual threshold such that the electrical stimulus does elicit sensations in the impaired limb.

In some implementations, the electrical stimulus activates sensory afferent cells of the spinal cord to increase the firing rate of intraspinal neural circuits and motoneurons innervating the impaired limb of the subject, thereby reducing the impairment.

In several implementations, the subject retains at least some residual activity of corticospinal tract neurons innervating the impaired limb, and may further retain at least some residual movement of the impaired limb.

In some implementations, the impairment is a proprioception impairment, for example, a reduced ability to detect force generated by and/or applied to the limb, and/or a reduced understanding of limb position and/or dynamics.

In some implementations, the neurostimulator is activated to apply the electrical stimulus in response to feedback from one or more sensors on the impaired limb.

The foregoing and other features and advantages of this disclosure will become more apparent from the following detailed description of several implementations which proceeds with reference to the accompanying figures.

### BRIEF DESCRIPTION OF THE FIGURES

FIG. 1 is a plan view of an eSCS system for use in some of the methods provided herein. An electrode array is implanted epidurally in the spinal cavity and proximate to dorsal rootlets and/or dorsolateral spinal cord adjacent to the dorsal rootlets, or proximate to the dorsal root ganglia (DRG), of sensory neurons innervating an arm of the patent. The lead is connected to an implanted neurostimulator. An external control unit is used to control the neurostimulator.

FIG. 2 is a peripheral view of the spinal cord and spinal nerves.

FIG. 3 is a cross-sectional view from the transverse plane of the spinal cord and spinal nerves.

FIG. 4A is a cross-sectional view from the sagittal plane of the upper spine and related structures illustrating placement of an electrode array implant as utilized in some of the disclosed methods for treating impairment of an upper limb due to neurological disorder or injury. As illustrated, a percutaneous lead containing an array of electrodes is implanted in the epidural at the dorsolateral spinal cord adjacent to the C3-T1 nerve roots. The lead connector exits the spinal cavity and is routed to the implanted neurostimulator.

FIG. 4B is a cross-sectional view from coronal plane of the cervical spine and related structures illustrating placement of an electrode array implant as utilized in some of the disclosed methods for treating impairment of an upper limb due to neurological disorder or injury. As illustrated, a percutaneous lead containing an array of electrodes is implanted in the epidural space at the dorsolateral spinal cord adjacent to sensory nerve root.

FIG. 5 is an expanded cross-sectional view of the upper spine and related structures illustrating the independently controllable electrodes of electrode array implant.

FIG. 6. Illustration of an exemplary cervical eSCS neurostimulation system for use in a patient with motor impairment of the left arm. The spinal lead contains an array of electrodes and is implanted epidurally in the spinal cavity and proximate to dorsal rootlets, and/or dorsolateral spinal cord adjacent to the dorsal rootlets, of sensory neurons innervating the left arm of the patent. The lead is connected to an implanted neurostimulator. An external control unit is used to control the neurostimulator. Additionally, electromyography (EMG) sensors are positioned on the left arm of the patient. The EMG sensors signal the implanted neurostimulator to apply a stimulus in response to detected electrical activity in the left arm of the patient.

FIGS. 7A-7D. Experimental framework and stimulation specificity. (FIG. 7A) Schematic of the experimental platform and paradigm. While participants performed an upper limb motor task, wireless electromyographic (EMG) activity from muscles of the arm and hand were measured. Electrical stimulation was delivered to the cervical spinal cord via two 8-contact leads (Rostral, R; Caudal, C) implanted in the cervical spinal cord. Stimulation through selected contacts simultaneously was controlled via percutaneous connections using an external stimulator. (FIG. 7B) X-rays of both participants showing the location of the contacts of the Rostral (light grey) and Caudal (dark grey) leads with respect to the midline. (FIG. 7C) Location of the motoneurons of arm and hand muscles in the human spinal cord in relation to spinal segments and vertebrae. (FIG. 7D) Graphical representation of muscle activation obtained by stimulating through selected contacts (labelled in red on the left of each human figurine). Each human figurine represents the front view (left half) and back view (right half) of arm muscles (See also FIG. 13). Each muscle is colored with a color scale (on the left) representing the normalized peak-to-peak amplitude of EMG reflex responses obtained during 1 Hz stimulation at the stimulation amplitude indicated on the left. Peak-to-peak values for each muscle are normalized to the maximum value obtained for that muscle across all contacts and all current amplitudes. On the left, MRI of each participant is shown with segmented lesion in red.

FIGS. 8A-8G. SCS immediately improves strength. (FIG. 8A) examples of single synchronized raw traces for torques and EMGs signals during isometric maximum voluntary contractions for extension (SCS01, left) and flexion (SCS02, right) of the elbow in the HUMAC® NORM™, (see panel FIG. 8G). (FIG. 8B) quantification of the root mean square value of EMG traces with and without stimulation during isometric elbow extension (SCS01) and flexion (SCS02) (FIGS. 8C, 8D, 8E) quantification of isometric torques during single joint flexion and extension for SCS01 and SCS02 at shoulder, elbow and wrist (FIG. 8F) quantification of isometric grip-strength measured with a hand-held dynamometer with and without stimulation. (FIG. 8G) schematic of the isometric torque test (wrist configuration in the example) in the HUMAC® NORM™. Statistics all quantifications are reported using box-plots. Median values are represented by circles. Inference on mean differences is performed by bootstrapping the n=5 repetitions obtained for each measurement, with n=10,000 bootstrap samples; * difference is outside the resulting 95% confidence interval.

FIGS. 9A-9E. SCS immediately improves arm kinematics. (FIG. 9A) schematic of the experimental set-up for planar reach out tasks using the KINARM. (FIG. 9B) Examples of raw endpoint trajectories for SCS01 in the reach out task. No stimulation on the left and during stimulation on the right. Inset shows inability to reach central target with no stimulation. Solid lines are reach trajectories and dashed lines represent pull trajectories. Darker lines represent average trajectories, shaded lines represent single trajectories. (FIG. 9C) Quantification of kinematic features, movement smoothness (velocity peaks) and time to reach target in s. Center target could not be calculated for no-stim condition because SCS01 did not complete the task. (FIG. 9D) Examples of raw endpoint trajectories for SCS02 in the reach out task. SCS02 was tasked to reach beyond the third horizontal line to complete the task. Reach and pull trajectories are represented in separate plots. (FIG. 9E) Quantification of kinematic features for SCS02, Reach time (equivalent to time to target in SCS01), Maximum reached distance and elbow angle excursion (max-min) are reported for no-stim (dark grey) and stim condition (light grey). Statistics all quantifications are reported using box-plots. Median values are represented by circles. Inference on mean differences is performed by bootstrapping the n=6 (center out) or n=5 (open-ended reaching) repetitions obtained for each measurement, with n=10,000 bootstrap samples; * difference is outside the resulting 95% confidence interval.

FIGS. 10A-101. SCS improves function. (FIGS. 10A-10C) Frame captures from videos showing improved functional abilities of different simulated activities of daily living: drawing a spiral, reaching and grasping a soup can, opening a lock for SCS01. Left no stimulation, right with stimulation. (FIG. 10D) picture report frames from video of SCS02 performing a modified “Hanoi tower” task in which she was task to move a hollow cylinder from a base pole to another. Left no stimulation, right with stimulation. (FIGS. 10E, 10F) representative pictures and quantification of task performances for SCS01 box and blocks and 3D fast reaching task performed on multiple days. (FIG. 10G) picture of the 3D reaching task using the ARMEO® Power for SCS02 and relative task performance on multiple days. (FIG. 10H) Fugl-Meyer assessment at different time points for SCS01 and SCS02 including 4-weeks post-study. (FIG. 101) normalized spasticity level obtained by averaging Modified Ashworth Score at each joint for SCS01 (dark grey) and SCS02 (light grey). Statistics all quantifications are reported using box-plots. Median values are represented by circles. Inference on mean differences is performed by bootstrapping the n=5 repetitions obtained for each measurement, with n=10,000 bootstrap samples; * difference is outside the resulting 95% confidence interval.

FIGS. 11A-11C. Lesion characterization and Lead position over time. (FIG. 11Aa) sagittal, coronal, and axial T1-weighted MRI 2D projections for SCS01 and SCS02. The segmented lesion is shown in red for both participant. R indicates the Right hemisphere. (FIG. 11B) High-definition fiber tracking of the corticospinal tract (CST) for SCS01 and SCS2. Colored fibers represent estimated CTS axons from the affected (right) and unaffected (left) hemisphere. Significant reduction in number of tracked fibers in the right hemisphere is clear in both participants in consequence of the stroke. (FIG. 11C) Repeated X-rays for SCS01 (left) and SCS02 (right) showing the position of the spinal leads. The red lines mark the same anatomical location across the X-rays to facilitate interpretation. Minimal displacement occurred after initial implantation.

FIGS. 12A-12E. SCS parameters set using a custom-built controller. (FIG. 12A) An image of the stimulator (DS8R, left) and 1-to-8 channel multiplexer (D188, right) used to deliver stimulation pulses. (FIG. 12B) An overview of the control scheme used to deliver patterns of stimulation. A PC running a (FIG. 12C) MATLAB based GUI communicated with a microcontroller using a custom (FIG. 12D) communication protocol over a virtual serial port. The microcontroller's firmware delivered pulse triggers and amplitude control signals to the stimulator as well as an 8 bit parallel channel selection signal to the multiplexer in order to control pulse timing, amplitude, and output channel. Current was delivered from the stimulator through the multiplexer and ultimately to the selected electrode on the implanted spinal array. (FIG. 12C) The GUI interface allowed for configuring all stimulation parameters including active channels, stimulation frequency, pulse train duration (or continuous), pulse train latency, and stimulation amplitude for each active channel. Once configured, stimulation was initiated or terminated via the software interface. The software also allowed for rapid changes in either global stimulation frequency (nudge frequency) or channel amplitude (nudge amplitude). (FIG. 12D) A custom command protocol layer was developed on top of a UART serial interface to enable communication between the GUI and microcontroller. Each packet from the master (PC) to the slave (microcontroller) comprised a 1 byte packet length, 1 byte command, and 0-6 bytes of payload. A payload comprised a 1 byte parameter (to be read or written), a 1 byte channel number (when appropriate), and the value to be written (when ‘write’ command was used). Microcontroller response packets comprised a 1 byte packet length, 1 byte command echo, 0-32 bytes of payload (used to return parameter values during ‘read’ command), and a 1 byte success flag. (FIG. 12E) The microcontroller firmware allowed for pseudo-synchronous stimulation across multiple channels by interleaving pulses on all active channels. A delay of at least 1 ms between each pulse allowed enough time for the multiplexer to fully switch channels. The same pattern of pulses was delivered every period as defined by the stimulation frequency. Each channel could also be configured to deliver a single pulse, a pulse train with finite duration and/or latency, continuous stimulation, or a ‘recruitment curve’ in which the amplitude was gradually increased for successive pulse trains of specified length.

FIG. 13. Muscle recruitment curves. Shown are the recruitment curves obtained with stimulation at 1 Hz at increasing current amplitude for 11 arm and hand muscles: TRAP: trapezius, A, P, M DEL: anterior, posterior and medial deltoid respectively, BIC: biceps, TRI: triceps, EXT: Extensor carpi, FLX: flexor carpi, PRO: pronator teres, ABP: abductor pollicis, and ADM: abductor digiti minimi. Below each set of recruitment curves the graphical representation of the muscle activation obtained at the amplitude indicated on the left of each human figurine is shown. Interpretation of human figurines is reported in the bottom right. Normalized peak-to-peak amplitude of EMG reflex responses obtained at the stimulation amplitude is indicated on the left. Peak-to-peak values for each muscle are normalized to the maximum value obtained for that muscle across all contacts and all current amplitudes.

FIG. 14. Frequency dependent suppression. To demonstrate that SCS recruits arm and hand muscles via direct activation of the primary afferents stimulation at multiple frequencies was performed. The figure reports the spinal reflexes obtained when stimulating at 1, 5, 10 and 20 Hz from multiple contact and multiple muscle. Each plot on the top shows the normalized peak-to-peak reflex amplitude as a function of frequency showing in the muscles that respond to the specific contact substantial frequency dependent suppression at stimulation frequencies greater than 10 Hz. On the bottom, raw EMG traces that show the classic phenomenon are shown. At 5 Hz each pulse of stimulation corresponds to a clear evoked potential in the EMG albeit amplitude slightly diminishes at each pulse. At 10 Hz, modulation of peak-to-peak amplitudes becomes more evident, at 20 Hz almost complete suppression of EMG evoked responses subsequent to the first is shown. Example is taken from Pronator muscles, contact 1C, (highlighted in darker grey in the top panel).

FIGS. 15A-15B. Optimized continuous stimulation protocols. Stimulation protocol used to achieve maximum assistive benefit for SCS01 (FIG. 15A) and SCS02 (FIG. 15B). (FIG. 15A) For SCS01, contacts 1R and 8R on the rostral lead and 7C on the caudal lead were simultaneously and continuously activated at a fixed 60 Hz frequency and 200 μs pulse width. These electrodes corresponded shoulders and biceps (1R); triceps, extensors, and hand opening (8R); and hand grasp (7C). Amplitudes were changed daily based on participant preference and were set to 2.4-2.6 mA (1R), 2.1-2.7 mA (8R), and 3.3-6.2 mA (7C). (FIG. 15B) For SCS02, contacts 1R on the rostral lead, and 1C, 5C, and 8C on the caudal lead were simultaneously and continuously stimulated. These electrodes corresponded to muscles related to shoulder support (1R); elbow flexion (1C); elbow extension and wrist flexion (5C); and hand grasp (8C). Contacts 1R and 1C were stimulated at 50 Hz while 5C and 8C were stimulated at 100 Hz all at a fixed pulse width of 400 μs. A reduced frequency was used on contacts corresponding to elbow flexion to bias the assistive benefit of stimulation toward elbow extension. Multi-frequency stimulation was achieved by skipping every other period of a 100 Hz stimulation protocol on channels stimulating at 50 Hz.

FIGS. 16A-16C. SCS improves arm kinematics supplementary metrics. (FIG. 16A) Effect of stimulation frequency shown for SCS01 and SCS02. In SCS01, quantification of isometric torques during single joint flexion and extension is shown for the elbow during no stim, 20 Hz, 40 Hz, and 60 Hz. In SCS02, maximum reached distance and elbow angle excursion (max-min) are reported during reach and pull of the reach-out task for no stim, 20 Hz, 40 Hz, and 60 Hz. Raw endpoint trajectories for SCS02 are shown in the reach out task during no stim, 20 Hz, 40 Hz, and 60 Hz, here SCS02 was tasked to reach beyond the third horizontal line to complete the task. Reach and pull trajectories are represented in separate plots. (FIG. 16B) Quantification of kinematic features for SCS01, path length for completed reach and pull of three targets in cm and variance of the path between trials are reported for no-stim (dark grey) and stim condition (light grey). Center target could not be calculated for no-stim condition because SCS01 did not complete the task. (FIG. 16C) Quantification of kinematic features for SCS02, movement smoothness (velocity peaks) and path length in cm for reach and pull separately are reported for no-stim (dark grey) and stim condition (light grey). The distribution of deviations from the mean path trajectory is shown in cm (equivalent to variance in SCS01). Inference on mean differences is performed by bootstrapping the n=5 repetitions obtained for each measurement, with n=10,000 bootstrap samples; * difference is outside the resulting 95% confidence interval.

FIGS. 17A-17E. Optimized SCS leads to best improvement. (FIG. 17A) Quantification of isometric torques during single joint flexion and extension of the elbow during no stim (dark grey), non-optimal stim (*), and optimal stim (**) for SCS01. (FIG. 17B) Quantification of performance for three targets of the center-out task during no stim (dark grey), non-optimal stim (*), and optimal stim (**) normalized from 0 (SCS02 never reached target) and 1 (SCS02 reached target in all trials). n=3 (FIG. 17C-FIG. 17E) Raw endpoint trajectories by SCS02 for three targets of the center-out task during no stim (dark grey), non-optimal stim (light grey), and optimal stim (medium grey). Inference on mean differences for (FIG. 17A) were performed by bootstrapping the n=5 repetitions obtained for each measurement, with n=10,000 bootstrap samples; * difference is outside the resulting 95% confidence interval.

FIGS. 18A-18I. Muscle activation pattern during planar movement. (FIG. 18A) Muscle label abbreviation used in the figure (FIG. 18B) Kinematic trajectories during planar center-out task for two different targets (left and center) for stimulation off and on condition. The inset block shows the inability of SCS01 to reach to the center target without stimulation. (FIG. 18C) EMG signals for the left target during reach and pull phase without and with stimulation (FIG. 18D) synergy vector (c) for left target corresponding to the increasing timeseries synergy activation. (FIG. 18E) EMG signals for the center target during reach and pull phase for the center target without and with stimulation. (FIG. 18F) Synergy vector for the center target with (light grey) and without stimulation (dark grey) for reach and pull phase. (FIG. 18G) Kinematic trajectories for reaching-out task with and without stimulation for reach (solid line) and pull phase (dashed line) (FIG. 18H) Muscle activity with and without stimulation during reach and pull phase for planar reaching-out task for SCS02. (FIG. 18I) Synergy vector corresponding to the reach and pull phase of the movement with (light grey) and without (dark grey) stimulation.

FIGS. 19A-19C. Position matching proprioception task. This task evaluates the participants ability to locate a precise location without vision, relying on their upper limb proprioception only. The participant has to reach two different targets (West & Northwest locations indicated by the gray dot at the end of the dotted lines leading to these positions). The kinematics of their movements are measured using a robotic platform. Thin traces represent individual trials, thick traces indicated average trajectory, and the circles represent the final position reached. For all images, dark grey represents the performance with stimulation off and light grey represents stimulation on. The shaded ovals represent the variability across repetitions. A smaller oval shape means a more consistent reaching location over repetitions. Trials were performed over three days, day 1 (FIG. 19A), day 2 (FIG. 19B), and day 3 (FIG. 19C).

FIG. 20. Body representation proprioception task. This task evaluates the participants' ability to recognize their upper limb part location in a static position. The specific body locations the participants were asked to locate were the index finger, ring finger, inner wrist, outer wrist, and elbow. While the participant was blindfolded, the impaired arm was occluded by concealing it under an opaque screen or box, so that the participant has no visual indication of the arm's position. Next, the participants were asked to focus on one of the body locations listed above and the examiner would move a pointer above the obscured arm. The pointer was visible by the participant. Once the participant felt the pointer was over the arm location being tested, the pointer location was recorded and compared to the actual position of that body location. FIG. 20A shows the actual locations of each body location and proprioceptively perceived locations reported by the participants and averaged over 5 repetitions without SCS. The plot of the left shows the same with SCS.

### DETAILED DESCRIPTION

### I. Introduction

Provided herein is a system and a method for treating impairment of a limb in a subject through electrical stimulation of sensory neurons innervating the impaired limb. In several implementations, the electrical stimulation is applied to the dorsal rootlets, dorsolateral spinal cord adjacent to the dorsal rootlets, or DRG, of sensory neurons innervating the impaired limb. For example, the electrical stimulus is applied with one or more electrodes controlled by a neurostimulator and implanted in the epidural space proximate to the dorsal rootlets or the dorsolateral spinal cord adjacent to the dorsal rootlets of sensory neurons innervating the limb of the subject, or the one or more electrodes are implanted proximate to DRG of sensory neurons innervating the limb of the subject. The impairment is a motor impairment and/or a proprioception impairment due to neurological disorder or injury (such as stroke). People who suffer from neurological disorders or injury caused by, for example, stroke, often suffer motor impairments as a result, and may be treated using the methods provided herein.

Here, motor impairment includes, but is not limited to, loss of control of the muscles or uncontrollable muscle tone know as spasticity, aberrant flexor synergies, or contractures. These motor deficits result in loss of independence, and difficulty performing everyday tasks of daily living. There are limitations to current modes of treatment. Physiotherapy often plateaus after 3-6 months without satisfactory rehabilitation. Functional electrical stimulation of the muscles using externally applied electrodes targets only a single joint and does not help alleviate muscle tone. Pharmaceutical interventions are designed only to help with reducing spasticity not improving motor control.

Prior investigation of spinal cord stimulation (SCS) for motor recovery focused on impairment caused by spinal cord injury (SCI) and more specifically on locomotion and leg movement utilizing epidural spinal cord implants (eSCS). A few studies explored the use of transcutaneous (non-invasive, outside the skin) spinal cord stimulation (tSCS) to recover upper limb function in SCI, however clinical results are not demonstrably better than classic technologies. Other work for using SCS focused on indications such as chronic pain, blood pressure regulation, and bladder control. However, given the large functional and anatomical differences between the lower and upper limbs as well as different disease origin, dynamics and manifestation between impairment due SCI compared to impairment due to neurological disorder or injury (e.g., stroke) it is not predictable that results obtained by eSCS on leg motor function for SCI could translate to eSCS for treatment of upper-limb function in neurological disorder (e.g., stroke).

In the context of SCS of the upper limbs, prior assessment is limited to transcutaneous stimulation. This is done by placing a stimulating electrode on the dorsal side of the neck above the cervical spinal cord and a return electrode on the ventral side of the neck. This form of stimulation has not demonstrated good efficacy in restoring movement to the upper limbs after SCI. tSCS and eSCS likely stimulate different physiological structures. tSCS is driving current through multiple layers of fat, muscle, skin, and bone besides stimulating the spinal cord without good specificity. eSCS can target the dorsal roots of the spinal cord with sub-segment resolution and is primarily stimulating the spinal tissue. Due to these differences, and the fact that tSCS has not demonstrated good efficacy, it was not predictable that eSCS would help recover motor function in the upper limbs of stroke patients.

eSCS has demonstrated some efficacy in lower limb motor recovery. Locomotion is a cyclical movement with the same sequence of muscles being activated in a regular temporal pattern. These patterns of activation are produced by specific circuits located in the spinal cord called Central Pattern Generators that produce locomotion. This is not the case in the upper limbs, where movements are much more heterogenous in order to interact with a huge variety of objects in many different contexts. This means that the neural underpinning of upper limb control is completely different from locomotion. Arm and hand control require sophisticated brain inputs to activate specific sub-circuits in the cervical region. There is no equivalent pattern generator that can produce large arm movement. This is why, to date, cervical SCS had largely failed. Indeed, approaches so far had employed the same strategies utilized for the lower limb, leading to unsatisfactory clinical outcomes despite months of physical therapy.

Compared to SCI, stroke presents itself as damage to the brain, not the spinal cord. The physiological consequences of each type of injury are therefore substantially different. For example, in SCI, it is possible and common for both motor and sensory pathways to be lesioned, within the spinal cord, changing the architecture of the connection between the spinal cord and brain in a unique way. This is compared to stroke, which is localized in the brain, while the spinal circuits remain intact, and therefore nervous system restructuring will be very different compared to SCI. Also, due to the rostro-caudal organization of the spinal cord, and SCI that results in loss of upper limb function would be in the cervical spinal cord, affecting the same exact tissue that would need to be the target of SCS. This is in contrast to loss of lower limb function in SCI which can present when damage is done anywhere rostral of the lumbosacral cord, leaving the spinal circuits innervating the lower limbs intact. In stroke, the cervical cord remains whole and therefore is a viable target for eSCS, but this is not the case in SCI, so any logic flowing from the SCI literature is not directly applicable to eSCS's indication for stroke.

Because of the brain origin of stroke, the vast majority of research in the last 80 years has focused on brain stimulation technologies. Therefore, the methods of the present disclosure provide a new and innovative approach for stroke therapy.

In contrast to prior studies, and as further described herein, in some implementations, an epidural array of electrodes is surgically implanted spanning the C3-T1 nerve roots and located in the epidural space proximate to the dorsal rootlets or the dorsolateral spinal cord adjacent to the dorsal rootlets of sensory neurons innervating a limb of the subject. Electrical stimulus is applied, for example, using a current or voltage-controlled stimulator using cathodic-first biphasic or monophasic charge balanced pulses with a cathodic pulse duration of about 200-500 μs, a pulse frequency between about 1 and 130 Hz, and a cathodic amplitude of about 0.01 μA to 12 mA. Stimulation may be applied continuously or in a phasic manner such that stimulation parameters change over the course of a given movement. By targeting the dorsal roots, the stimulation indirectly activates motor circuitry though sensory fibers entering the spinal cord. By activating the intermediate circuitry within the spinal cord, the stimulation can both assist in performing movements as well as reduce spasticity and other forms of contracture. By targeting multiple spinal segments simultaneously, the stimulation can provide support for multiple joints along the upper limb. The stimulation can be applied at or below the motor threshold such that the stimulation does not directly elicit movements but instead these movements can be controlled voluntarily by the user. Stimulation can therefore be helpful in assisting or strengthening voluntary movements that the user cannot perform without intervention. Continuous stimulation of three different sites simultaneously can permit support of the shoulder, elbow, wrist, and hand such that the user regains the ability to voluntarily perform movements without assistance with marked improvements in strength, range of motion, dexterity, and/or and function in all these joints

The disclosed methods can be used as a daily assistive therapy to help patients perform everyday tasks and regain independence. The disclosed methods can also be used as a tool during therapy to help a patient perform movements during a therapeutic session that they could not otherwise perform, thus reducing muscle atrophy, gaining muscle strength, and allowing for more advanced physiotherapy sessions that will have better clinical outcomes.

### II. Acronyms

CST Corticospinal tract
DRG Dorsal root ganglia
EMG Electromyography
SCI Spinal cord injury
SCS Spinal cord stimulation
eSCS Epidural spinal cord stimulation
tSCS Transcutaneous spinal cord stimulation

### III. Summary of Terms

Unless otherwise noted, technical terms are used according to conventional usage. As used herein, the term “comprises” means “includes.” Although many methods and materials similar or equivalent to those described herein can be used, particular suitable methods and materials are described below. In addition, the materials, methods, and examples are illustrative only and not intended to be limiting. The scope of the claims should not be limited to those features exemplified. To facilitate review of the various implementations, the following explanations of terms are provided:

About: As used herein, the term “about” refers to an approximation of a qualitative or quantitative measurement. Whether the measurement is qualitative or quantitative should be clear from its context. With regard to quantitative measurements, “about” refers to plus or minus 5% of a reference value. For example, “about” 100 mA refers to 95 mA to 105 mA.

Dorsal rootlets: Small branches of a root of a sensory neuron that emerges from the posterior spinal cord and travels to the DRG.

Dorsolateral spinal cord: A region on the exterior surface of the spinal cord located between the dorsal midline and the point of entry of the dorsal rootlets into the main cord.

Electrical stimulus: The passing of various types of current or voltage selectively through one or more electrodes to a target location in a subject (for example, specific areas of the dorsolateral spinal cord).

Electrode: An electric conductor through which an electric current can pass. An electrode can also be a collector and/or emitter of an electric current. In some implementations, an electrode is a solid and comprises a conducting metal as the conductive layer. Non-limiting examples of conducting metals include noble metals and alloys, such as stainless steel and tungsten. An array of electrodes refers to a device with at least two electrodes formed in any pattern. A multi-channel electrode includes multiple conductive surfaces that can independently activated to stimulate or record electrical current.

Implanting: Completely or partially placing a neurodevice (such as a neurostimulator connected to an electrode array within a subject, for example, using surgical techniques. A neurodevice is partially implanted when some of the device or neurostimulator reaches, or extends to the outside of, a subject. A neurodevice can be implanted for varying durations, such as for a short-term duration (e.g., one or two days or less) or for long-term or chronic duration (e.g., one month or more).

Motor impairment: The partial or total loss of function of the muscles of a body part, for example, legs, feet, arms, hands, fingers, neck, and trunk (e.g., respiratory muscles). Particular motor impairments include loss of muscle strength, partial paralysis (paresis), loss of dexterity (such as hand-finger movement), and uncontrollable muscle tone (spasticity). In some aspects, the motor impairment is the partial loss of function of one or both shoulder and/or arms and/or hands due to stroke. In other aspects, the motor impairment is the partial loss of function in the lower limbs due to cerebral palsey. These motor deficits result in loss of independence, and difficulty performing everyday tasks of daily living.

Motor threshold: The minimum spinal stimulation intensity that can produce a motor output of a given amplitude from a muscle at rest (RMT) or during an active muscle contraction (AMT).

Proprioception impairment: The partial or total loss of the sense of self-movement, force, and/or position, of a body part, for example, legs, feet, arms, hands, fingers, neck, and trunk. In some aspects, the proprioception impairment is loss of body representation and understanding of arm position due to stroke. In other aspects, the proprioception impairment is loss of arm velocity information caused by ALS.

Neural signal: An electrical signal originating in the nervous system of a subject. “Stimulating a neural signal” refers to application of an electrical current to the neural tissue of a subject in such a way as to cause neurons in the subject to produce an electrical signal (e.g., an action potential). An extracellular electrical signal can, however, originate in a cell, such as one or more neural cells. An extracellular electrical signal is contrasted with an intracellular electrical signal, which originates, and remains, in a cell. An extracellular electrical signal can comprise a collection of extracellular electrical signals generated by one or more cells.

Neurological disorder or injury: A disease or injury of the brain that leads to Motor Impairment and/or Proprioception Impairment of one or more limbs in a subject. Neurological disorders could include neurodegenerative disorders such as motorneuon diseased or muscular dystrophy diseases such as amyotrophic lateral sclerosis or Duchenne motor disorder. Neurological injury could include stroke, cerebral palsy, or traumatic brain injury.

Neurostimulator: A current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, and on-time and off-time. The controlled electrical pulse is delivered through one or more electrodes (for example, leadless electrode(s), or electrode(s) located at the end of a lead, a thin insulated wire) configured to apply the electrical stimulus to target tissue of a subject. A neurostimulator may comprise at least one multiple contact lead. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback from a subject's movement).

Perceptual threshold: The minimum applied electrical stimulation intensity necessary for a conscious human to be aware of a particular sensation caused by the electrical stimulation.

Sensory neurons: Also known as afferent neurons, sensory neurons are nerve cells within the peripheral nervous system responsible for converting stimuli from the environment of the neuron into internal electrical impulses and transmitting the impulse to the central nervous system.

Stroke: The sudden death of brain cells due to a lack of oxygen when the blood flow to the brain is impaired by blockage or rupture of an artery to the brain. Ischemic stroke refers to a condition that occurs when an artery to or in the brain is partially or completely blocked such that the oxygen demand of the tissue exceeds the oxygen supplied. Ischemic stroke is by far the most common kind of stroke, accounting for about 80% of all strokes. Deprived of oxygen and other nutrients following an ischemic stroke, the brain suffers injury as a result of the stroke. The most common cause of ischemic stroke is narrowing of the arteries in the neck or head. This is most often caused by atherosclerosis, or gradual cholesterol deposition. If the arteries become too narrow, blood cells may collect in them and form blood clots (thrombi). These blood clots can block the artery where they are formed (thrombosis), or can dislodge and become trapped in arteries closer to the brain (embolism). Another cause of ischemic stroke is blood clots in the heart, which can occur as a result of irregular heartbeat (for example, atrial fibrillation), heart attack, or abnormalities of the heart valves. While these are the most common causes of ischemic stroke, there are many other possible causes. Examples include use of street drugs, traumatic injury to the blood vessels of the neck, or disorders of blood clotting. Hemorrhagic stroke is another kind of stroke that results from an accumulation of blood in or around the brain, such as from a ruptured blood vessel. Hemorrhages in the brain can be caused by a variety of disorders that affect the blood vessels, such as long-term high blood pressure and cerebral aneurysms (a week or thin spot on a blood vessel wall).

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects.

Therapeutically effective amount: An amount sufficient to provide a beneficial, or therapeutic, effect to a subject or a given percentage of subjects. Therapeutically effective amounts of a treatment can be determined in many different ways, such as assaying for a reduction in a disease or condition (such as motor impairment and/or proprioception impairment due to neurological disorder or injury). Therapeutic treatments can be administered in a single application, or in several applications (e.g., chronically over an appropriate period of time). However, the effective amount can be dependent on the source applied, the subject being treated, the severity and type of the condition being treated, and the manner of administration.

Treating or treatment: With respect to disease or condition (e.g., motor impairment and/or proprioception impairment due to neurological disorder or injury), either term includes (1) preventing the disease or condition, e.g., causing the clinical symptoms of the disease or condition not to develop in a subject that may be exposed to or predisposed to the disease or condition but does not yet experience or display symptoms of the disease or condition, (2) inhibiting the disease or condition, e.g., arresting the development of the disease or condition or its clinical symptoms, or (3) relieving the disease or condition, e.g., causing regression of the disease or condition or its clinical symptoms.

IV. Stimulation of Sensory Neurons to Treat Motor Impairment and/or Proprioception Impairment

Provided herein are methods for treating a subject (for example, a human subject) with motor impairment and/or proprioception impairment of a limb due to neurological disorder or injury. The methods may be utilized to treat (i.e., prevent, ameliorate, suppress, and/or alleviate) the motor impairment and/or proprioception impairment due to the neurological disorder or injury. The method includes application of a therapeutically effective amount of an electrical stimulus to sensory neurons innervating the limb of the subject with the motor impairment and/or proprioception impairment. The electrical stimulus is applied with one or more electrodes controlled by a neurostimulator. In several implementations, the impairment is impairment of voluntary movement of the limb.

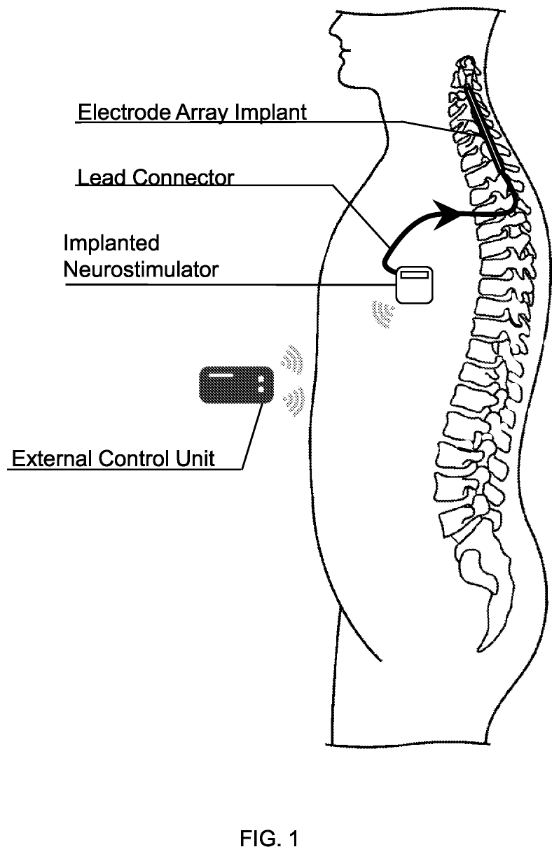

An implementation of the disclosed method is illustrated in FIG. 1. In the figure, placement of the electrode array is proximate to, e.g., resting upon, the spinal cord area to be stimulated (in this case, the cervical spinal cord). The neurostimulator is generally implanted in a surgically-made pocket either in the abdomen or above the buttocks, though other locations of the subject's body are also possible. The neurostimulator is connected to the electrode array via one or more lead connectors. The lead connectors facilitate locating the neurostimulator away from the electrode array implant. As shown, an external control unit is used to control the implanted neurostimulator and program stimulation parameters.

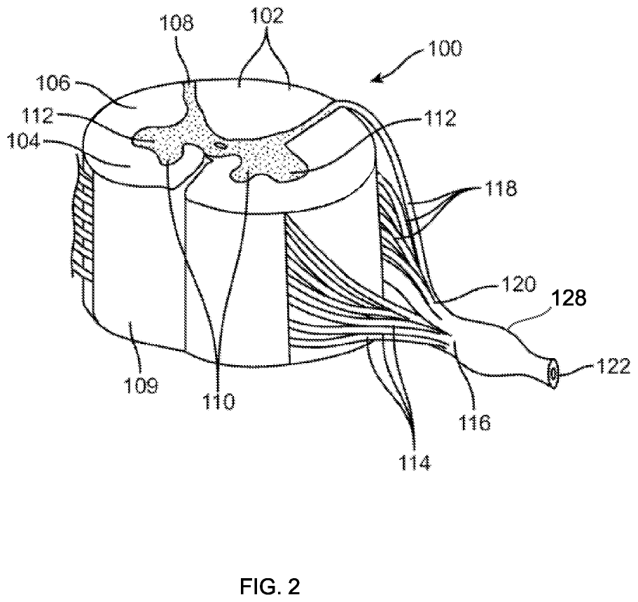

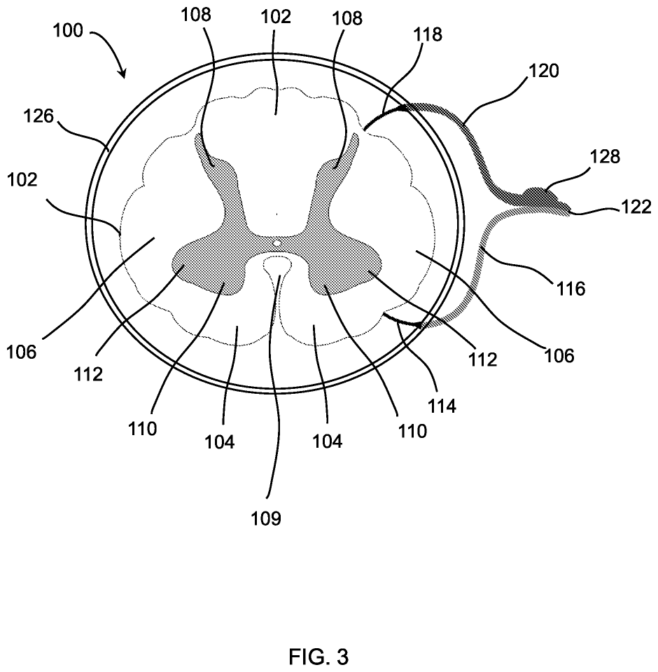

FIGS. 2 and 3 provide additional detail on the anatomical region targeted for stimulation in the methods provided herein. The spinal cord 100 is divided into three functional columns: the dorsal column 102, the ventral column 104, and the lateral columns 106. Similarly, the butterfly-shaped gray matter of the spinal cord 100 is divided into the dorsal horn 108, the ventral horn 110, and the lateral horn 112. A ventral median fissure 109 divides the spinal cord 100 into two lateral halves. The spinal cord 100 is enclosed by a dura mater 126, with an epidural space surrounding the dura mater.

A group of motor nerve rootlets (ventral root nerve fibers) 114 branch off of the ventral horn 110 and combine to form the ventral root 116. Similarly, a group of sensory nerve rootlets (dorsal root nerve fibers) 118 branch off of the dorsal horn 108 and combine to form the dorsal root 120, which extends to the DRG 128. The dorsal root 120 and the ventral root 116 combine to form the spinal nerve 122, which innervates peripheral regions (e.g., arms, legs, etc.) of the patient's body.

In the methods provided herein, the electrical stimulus is applied to sensory neurons innervating the limb of the subject. Accordingly, the one or more electrodes used to apply the electrical stimulus are implanted in the subject within a suitable distance of the one or more sensory neurons innervating a limb of the subject with the with the motor impairment and/or proprioception impairment. The sensory neurons are stimulated, for example, at the dorsal roots, dorsal rootlets, or DRG, or within the dorsal or ventral horn, via electrodes implanted at a suitable location, such as epidural, subdural, or intraspinal implants.

In a preferred implementation, the electrical stimulus is applied to dorsal roots, dorsal rootlets, or DRG, of sensory neurons innervating the impaired limb of the subject. Accordingly, the one or more electrodes used to apply the electrical stimulus are implanted in the subject within a suitable distance of the dorsal roots, dorsal rootlets, and/or DRG of the sensory neurons innervating a limb of the subject with the with the motor impairment and/or proprioception impairment. Typically, the one or more electrodes are implanted in the epidural space at the dorsolateral aspect of the spinal cord and proximate to the dorsal roots or dorsal rootlets of the sensory neurons innervating the limb of the subject, or the one or more electrodes are implanted proximate to dorsal root ganglia of sensory neurons innervating the limb of the subject. As used herein, an electrode that is implanted “in the epidural space at the dorsolateral aspect of the spinal cord and proximate to the dorsal roots or dorsal rootlets of the sensory neurons innervating the limb of the subject” is an electrode that is within sufficiently close distance to the dorsal roots or dorsal rootlets of the sensory neurons innervating the limb of the subject to emit an electrical stimulus that stimulates the dorsal roots or dorsal rootlets without also stimulating neuronal tissue at the dorsal midline of the spinal cord. In some implementations, such an electrode is located in the epidural space between the point of entry of the dorsal rootlets into the main cord and a half-way point between the dorsal midline and the point of entry of the dorsal rootlets into the main cord. In several implementations, the electrodes are anchored into position to prevent or reduce migration, for example, by attachment to bony structures near the implantation site.

In some implementations, the one or more electrodes are not implanted adjacent to the lateral spinal cord.

Any appropriate method may be used to implant the electrodes of the neurostimulator at an appropriate anatomical location in the subject. In several implementations, the electrodes of the neurostimulator are tunneled percutaneously and secured in place with tape or suture in the subject. The electrodes may be steered laterally under fluoroscopic guidance to target the dorsal rootlets and the dorsolateral spinal cord, for example, using a stylet.

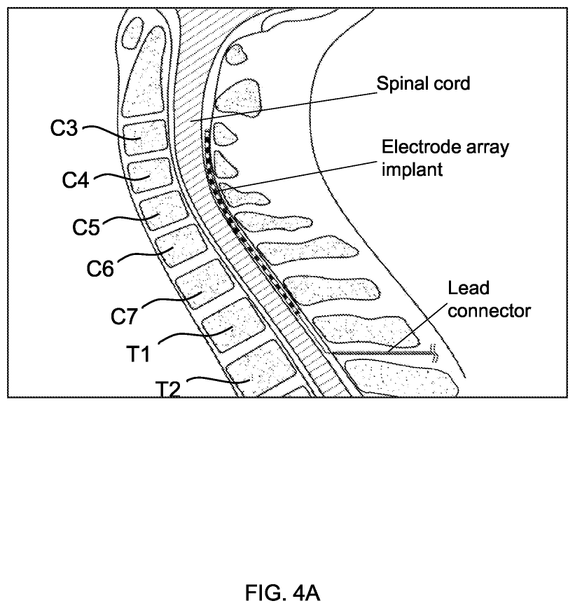

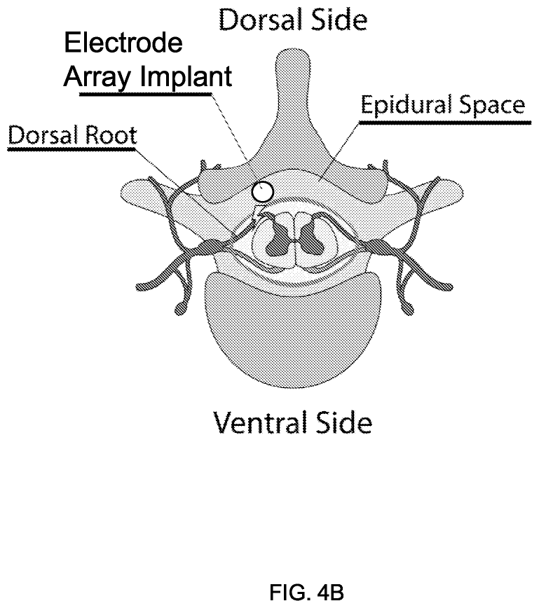

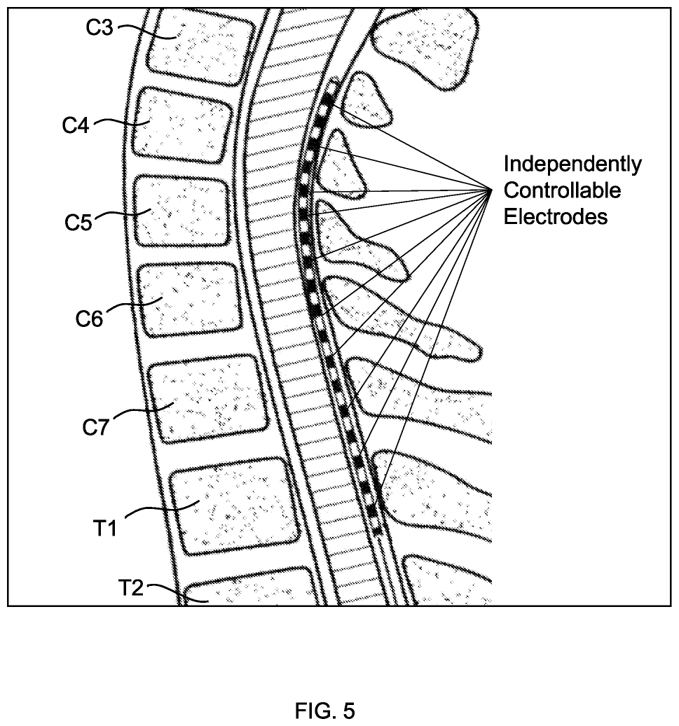

FIGS. 4A, 4B, and 5 further illustrate placement of the electrode array, in an implementation for treating impairment of an upper limb. As shown in FIG. 4A, a percutaneous lead containing an array of electrodes is implanted epidurally at the dorsolateral spinal cord adjacent to the C3-T1 nerve roots. The lead connector exits the spinal cavity and is routed to the implanted neurostimulator. FIG. 4B illustrates placement of the lead in the epidural space at the dorsolateral aspect of the spinal cord and adjacent to where the dorsal rootlets enter the main cord. FIG. 5 is an expanded view showing that the implanted percutaneous lead contains multiple independently controllable electrodes, allowing optimization of the stimulus signal to specific anatomical regions.

Applying the electrical stimulus to the sensory neurons increases the firing rate probability of spinal motoneurons innervating the limb of the subject with the motor impairment and/or proprioception impairment. Without being bound by theory, it is believed that application of the electrical stimulus to sensory neurons directly recruits mono- and poly-synaptic excitatory pathways in the spinal cord, which indirectly increases the membrane potential and firing rate probability of the spinal motoneurons innervating the impaired limb of the subject, which reduces the impairment of the limb.

In some implementations, application of the therapeutically effective amount of the electrical stimulus over time (for example, on a daily basis for at least a month or at least 6 months or at least a year) leads to a reduction in the impairment even in the absence of the stimulation. In some implementations, application of the therapeutically effective amount of the electrical stimulus over time (for example, on a daily basis for at least a month or at least 6 months or at least a year) increases the number of active motoneurons innervating the impaired limb of the subject, which reduces the impairment even in the absence of the stimulation.

Any appropriate subject with or at risk of a motor impairment and/or proprioception impairment due to neurological disorder or injury can be treated with the method provided herein. The impairment can be in a limb of the upper or lower body, such as an above or below the elbow, or an above or below the knee, or including the entire arm or leg.

In some implementations, the subject retains at least some residual activity of corticospinal tract neurons innervating the impaired limb. In some implementations, the subject retains at least some residual movement of the impaired limb.

The method can be in initiated at any time post-onset of the neurological disorder or injury (such as stroke). In some implementations, the method provided herein is implemented as soon as possible following the occurrence of the neurological disorder or injury in the subject, such as soon as possible following stroke in the subject that results in neurological disorder or injury that leads to motor impairment and/or proprioception impairment, so as to maximally arrest cortical changes subsequent to the injury. In some implementations, the method provided herein is implanted a long time after the occurrence of the neurological disorder or injury, such as more than one year or more than five years following the occurrence of the neurological disorder or injury.

In some implementations, the motor impairment and/or proprioception impairment is due to stroke, such as ischemic stroke or hemorrhagic stroke. In some implementations, a subject with stroke is selected for treatment. In some implementations, the motor impairment and/or proprioception impairment is due to a neurological disorder, such as amyotrophic lateral sclerosis or Duchenne motor disorder. The method can be in initiated at any time post-onset of the motor impairment in the subject, or even in advance of detectable motor impairment in a patient at risk of motor impairment. In some implementations, the impairment is not caused by spinal cord injury.

Application of the therapeutically effective amount of the electrical stimulus to the subject treats at least one motor impairment and/or proprioception impairment due to the neurological disorder or injury in the subject. For example, application of the therapeutically effective amount of the electrical stimulus can result in a reduction in the loss of muscle strength, an increase in muscle strength, a reduction in muscle atrophy, a reduction in the loss of motor function, an increase in motor function; an increase in joint flexion,

In some implementations, the subject has reduced control of the impaired limb and application of the therapeutically effective amount of the electrical stimulus increases control of the impaired limb by at least 20% (such as at least 30%, at least 40%, at least 50%, at least 60%, at least 70%, at least 80%, at least 90%) relative to before the treatment as measured by any appropriate evaluation metric, such as a balance or strength metric (e.g., a sensory organization test).

In some implementations, the subject has reduced elbow movement (such as flexion and/or extension) in the impaired limb and application of the therapeutically effective amount of the electrical stimulus increases elbow torque of the impaired limb by at least 20% (such as at least 30%, at least 40%, at least 50%, at least 60%, at least 70%, at least 80%, at least 90%) relative to before the treatment as measured using any suitable instrument.

In some implementations, the subject has reduced shoulder movement (such as flexion, extension, abduction, and/or adduction) in the impaired limb and application of the therapeutically effective amount of the electrical stimulus increases wrist torque of the impaired limb by at least 20% (such as at least 30%, at least 40%, at least 50%, at least 60%, at least 70%, at least 80%, at least 90%) relative to before the treatment as measured using any suitable instrument.

In some implementations, the subject has reduced wrist movement (such as flexion, extension, abduction, and/or adduction) in the impaired limb and application of the therapeutically effective amount of the electrical stimulus increases wrist torque of the impaired limb by at least 20% (such as at least 30%, at least 40%, at least 50%, at least 60%, at least 70%, at least 80%, at least 90%) relative to before the treatment as measured using any suitable instrument.

In some implementations, the subject has reduced grip strength or hand dexterity in the impaired limb and application of the therapeutically effective amount of the electrical stimulus increases grip strength of the impaired limb by at least 20% (such as at least 30%, at least 40%, at least 50%, at least 60%, at least 70%, at least 80%, at least 90%) relative to before the treatment as measured using any suitable instrument.

In some implementations, the one or more electrodes are implanted in the epidural space at the dorsolateral aspect of the spinal cord and proximate to the dorsal roots or dorsal rootlets of the sensory neurons innervating the limb of the subject, or the one or more electrodes are implanted proximate to DRGs of sensory neurons innervating the limb of the subject. In some implementations, the one or more electrodes are implanted in the epidural space proximate to the dorsal rootlets of the one or more sensory neurons innervating the impaired limb of the subject. In some implementations, the one or more electrodes are implanted in the epidural space proximate to the dorsal roots of the one or more sensory neurons innervating the impaired limb of the subject. In some implementations, the one or more electrodes are implanted proximate to the DRG of the one or more sensory neurons innervating the body region with the motor impairment of the subject.

The electrodes can be implanted at any appropriate position along the spinal cord, depending on the limb of the subject affected. In some implementations, the method is used to treat motor impairment and/or proprioception impairment in a lower extremity. Lumbar T11, T12, L2, L3, L4, L5, and/or sacral S1 are known to contain sensory neurons receiving signals from the lower extremities and can be targeted for stimulation using the method provided herein. In some implementations, the method is used to treat motor impairment and/or proprioception impairment in an upper extremity. Cervical spinal segments C3, C4, C5, C6, C7, C8, thoracic T1 are known to contain sensory neurons receiving signals from the upper extremities and can be targeted for stimulation using the method provided herein.

In some implementations, the impairment is in an upper limb of the subject, such as an upper arm, a shoulder, an arm, a hand, and the one or more electrodes are implanted to apply an electrical stimulus to one or more sensory neurons of the C3-T2 (such as the C3-T1 nerve roots). For example, the one or more electrodes are implanted proximal to the DRG, or are implanted epidurally proximal to the dorsal rootlets or dorsal roots of one or more of the sensory neurons of the C3-T2 nerve roots (such as the C3-T1 nerve roots).

In some implementations, the impairment is in a lower limb of the subject, such as the lower back, a hip, a leg, an ankle, or a foot, and the one or more electrodes are implanted to apply an electrical stimulus to one or more sensory neurons of the T11-S1 nerve roots, for example, the one or more electrodes are implanted proximal to the DRG, or are implanted epidurally proximal to the dorsal rootlets or dorsal roots of one or more sensory neurons of the T11-S1 nerve roots.

The applied stimulus parameters can vary depending on the particular subject and desired outcome. In several implementations, the stimulus parameters are calibrated for the particular subject to be treated with the disclosed method. Stimulation parameters that may be modulated include stimulus amplitude, pulse duration, frequency, and number of pulses. In some implementations, recurring trains of stimulus pulses may be delivered to the anatomical targets. Further, the duration and frequency of stimulation can be varied as needed to optimize therapeutic outcome. In some implementations, the intensity of the stimulation (and any resulting sensation) can be calibrated to correlate with the level of improvement in motor impairment and/or proprioception impairment of the limb in the subject.

Thus, any suitable stimulation pattern may be used that treats the motor impairment and/or proprioception impairment in the subject. In some implementations, the electrical stimulus includes electrical pulses defined by parameters including, for example and without limitation, amplitude, pulse duration, and pulse frequency. Such electrical pulses may include charge-balanced pulses, such as cathodic-first biphasic or monophasic charge balanced pulses. In these and further implementations, the electrical stimulus may be a continuous electrical stimulus, or a periodic stimulus.

In particular examples, the electrical stimulus includes electrical pulses with an amplitude of about 0.01 μA to about 50 mA, such as about 10 μA to 10 mA, about 10 μA to about 1 mA, about 10 μA to about 100 μA, or about 100 μA to about 1 mA.

In particular examples, the electrical stimulus includes electrical pulses with pulse durations between about 40 μs and about 2 ms; for example, between 40 μs and 2 ms, between 100 μs and 2 ms, between 200 μs and 2 ms, between 300 μs and 2 ms, between 400 μs and 2 ms, between 500 μs and 2 ms, between 600 μs and 2 ms, between 700 μs and 2 ms, between 800 μs and 2 ms, between 800 μs and 2 ms, between 900 μs and 2 ms, between 1 ms and 2 ms, between 1.5 ms and 2 ms, between 80 μs and 1.5 ms, between 100 μs and 1.5 ms, between 200 μs and 1.5 ms, between 300 us and 1.5 ms, between 400 μs and 1.5 ms, between 500 μs and 1.5 ms, between 600 μs and 1.5 ms, between 700 μs and 1.5 ms, between 800 μs and 1.5 ms, between 800 μs and 1.5 ms, between 900 μs and 1.5 ms, between 1 ms and 1.5 ms, between 1.5 ms and 2 ms, between 80 μs and 1 ms, between 100 μs and 1 ms, between 200 μs and 1 ms, between 300 μs and 1 ms, between 400 μs and 1 ms, between 500 μs and 1 ms, between 600 μs and 1 ms, between 700 μs and 1 ms, between 800 us and 1 ms, between 800 μs and 1 ms, and between 900 μs and 1 ms.

In particular examples, the electrical stimulus includes a pulse frequency between about 0.01 Hz and about 50,000 Hz; for example, between about 1 Hz to about 10,000 Hz, between about 10 Hz and about 500 Hz, between about 10 Hz and about 300 Hz, between about 10 Hz and about 100 Hz; for example, between 20 Hz and 100 Hz, between 20 Hz and 90 Hz, between 20 Hz and 80 Hz, between 20 Hz and 70 Hz, between 20 Hz and 60 Hz, between 20 Hz and 50 Hz, between 20 Hz and 40 Hz, and between 20 Hz and 30 Hz.

In some implementations, the electrical stimulus includes electrical pulses having an amplitude of 10 μA to about 50 mA, a pulse duration of between about 40 μs and about 2 ms, and a pulse frequency between about 10 Hz and about 2000 Hz.

In some implementations, the electrical stimulus includes electrical pulses having an amplitude of 10 μA to about 20 mA, a pulse duration of between about 40 μs and about 1 ms, and a pulse frequency between about 10 Hz and about 300 Hz.

In some implementations, the electrical stimulus includes electrical pulses having an amplitude of 100 μA to about 10 mA, a pulse duration of between about 40 μs and about 500 μs, and a pulse frequency between about 10 Hz and about 1000 Hz.

In some implementations, the electrical stimulus includes electrical pulses having an amplitude of 10 μA to about 15 mA, a pulse duration of between about 40 μs and about 500 μs, and a pulse frequency between about 10 Hz and about 200 Hz.

In some implementations, the electrical stimulus includes electrical pulses having an amplitude of less than about 10 mA, a pulse duration of between about 80 μs and about 2 ms, and a pulse frequency between about 20 Hz and about 100 Hz.

In some examples, the electrical stimulus includes electrical pulses having an amplitude of between 0.5 mA and 5 mA, pulse durations between 80 μs and 200 μs, and a pulse frequency between 20 Hz and 80 Hz. For example, the electrical stimulus may include electrical pulses having an amplitude of between about 1.5 and about 3.5 mA, pulse durations between about 100 μs and about 200 μs, and a pulse frequency between about 40 Hz and about 80 Hz.

The electrical stimulus may be applied to the patient for any suitable amount of time needed to achieve a positive functional benefit for the patient.

In some implementations, a particular pattern of stimulation, which may be person-specific, will be more effective than others at treating the motor impairment and/or proprioception impairment. In some implementations, a pattern of signals approximating the train of signals received from a normal, innervated limb for communicating sensations of pressure, touch, joint movement, proprioception, and/or kinesthesia to the cortex is used. In some implementations, the neurostimulator may be programmed to optimize such stimulation patterns, or the choice of stimulation patterns may be controlled by the subject or a health care provider. For example, subject or health care provider may adjust the amplitude and frequency of signals, for example, and also may select which channel (i.e., electrode) transmits which signal, to optimize signal pattern.

In some implementations, the electrical stimulus comprises a stimulation pattern of a series of 2 to 5 pulses separated by inter-pulse intervals of about 0.5 ms to about 10 ms and wherein the series is repeated at a frequency of about 10 Hz to about 500 Hz. In some implementations, the electrical stimulus comprises a stimulation pattern of a series of 2 to 5 pulses separated by inter-pulse intervals of about 0.5 ms to about 10 ms and wherein the series is repeated at a frequency of about 200 Hz to about 500 Hz. In some implementations, the electrical stimulus comprises a stimulation pattern of a series of 3 pulses separated by inter-pulse intervals of about 5 ms and wherein the series is repeated at a frequency of about 30 Hz to about 100 Hz, and wherein the pulse duration is about 200 μs.

In particular implementations, the electrical stimulus is applied at or below the subject's motor threshold and/or at or below the subject's perceptual threshold. For example, the stimulation may be applied below the motor threshold and below the perceptual threshold; below the motor threshold, but at or above the perceptual threshold; or below the perceptual threshold, but at or above the motor threshold. In some implementations, application of the electrical stimulus does not induce paresthesia in the subject.

In some implementations, stimulation parameters are selected that elicit focal sensations of touch, pressure, joint movement, proprioception, and/or kinesthesia in the affected limb(s) in the subject. In other implementations, stimulation parameters are selected that are below (such as 10-50% below, for example, 10%, 20%, 30%, 40%, or 50% below) the threshold or for eliciting focal sensations of touch, pressure, joint movement, proprioception, and/or kinesthesia in the affected limb(s) in the subject.

In some non-limiting implementations, treating the patient with the therapeutically effective amount of the electrical stimulus increases inputs on the membrane of the spinal motoneurons by means of direct recruitment of sensory afferents from the electrical pulses, and leads to ion channel remodeling on the motoneuron membrane to increasing firing rate probability of spinal motoneurons innervating the impaired limb of the subject to reduce the motor impairment, including when the electrical stimulus is not applied to the patient.

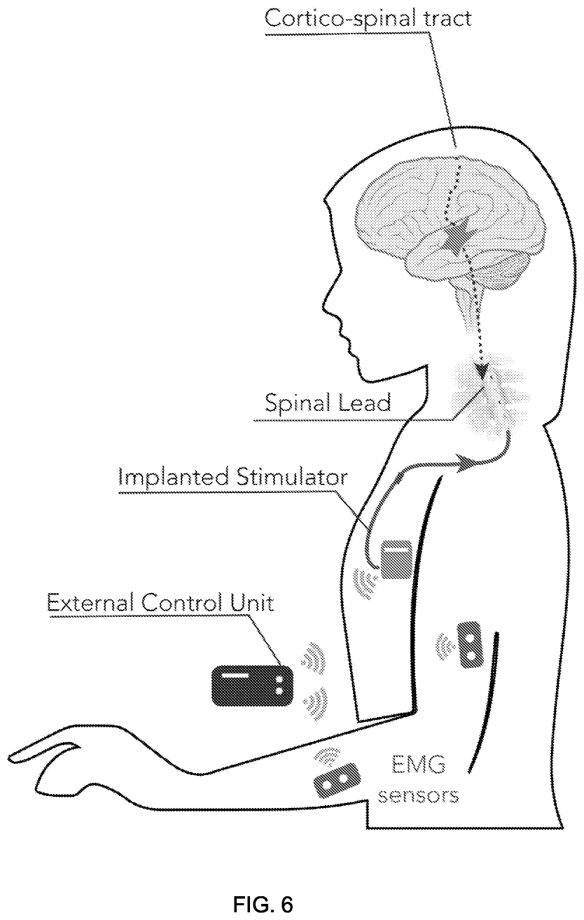

In some implementations, the disclosed methods include the use of an implanted neurostimulator that controls the stimulation (e.g., electrical stimulation via one or more implanted electrode(s)) according to parameters determined by feedback in a closed-loop system. One example is illustrated in FIG. 6, which shows an exemplary cervical eSCS neurostimulation system for use in a patient with motor impairment of the left arm. The spinal lead contains an array of electrodes and is implanted epidurally in the spinal cavity and proximate to the dorsal roots or dorsal rootlets of sensory neurons innervating the left arm of the patent. The lead is connected to an implanted neurostimulator. An external control unit is used to control the neurostimulator. Additionally, EMG sensors are positioned on the left arm of the patient. The EMG sensors signal the implanted neurostimulator to apply a pre-programmed electrical stimulus when muscle electrical activity is detected. In patients with residual motor activity, the EMG sensor will detect weak muscle activation and will signal the neurostimulator to apply a targeted electrical stimulus, which in turn will promote movement of the affected limb by the patient.

In particular implementations, the one or more electrodes and the neurostimulator comprise a daily assistive device that improves muscle weakness in an affected limb of the subject.

Stimulation of sensory afferents using implanted electrodes is an advanced neurosurgical procedure involving the implantation of one or more electrode(s) that deliver an electrical stimulus under the control of an externalized or implanted neurostimulator unit. Implantation of the electrode(s), and/or a neurostimulator in examples where the neurostimulator is not externalized, is typically performed by a clinical team including neurologists, neurosurgeons, neurophysiologists, pain management physicians, and other specialists trained in the assessment, treatment, and care of neurological conditions. Typically, following selection of an appropriate subject and determination of the target area of the subject to be stimulated, precise placement of at least one electrode in the area of the patient's sensory afferents (such as the dorsolateral aspect of the spinal cord) is carried out in an operating room setting, typically utilizing spinal cord imaging technology. After administration of local anesthesia, the subject undergoing electrode implantation experiences little discomfort, and may be kept awake during the implantation procedure to allow communication with the surgical team.

Some implementations herein employ an implant that includes one or more electrodes and/or neurostimulator implanted (e.g., fully or partially implanted) in the subject. Further implementations herein employ an implant that includes one or more magnets or optical fibers, and/or a neurostimulator implanted in the subject.

The implanted electrodes can have any form appropriate for stimulating neural signals in the dorsal roots or dorsal rootlets of one or more sensory neurons innervating a limb of a subject, and/or corresponding DRG. In some implementations, multi-channel electrode arrays are used. For example, the individual channels of the electrode can be calibrated to generate neural signals at a desired location in the subject (such as neural signals that induce sensations of pressure or touch in the limb with motor impairment and/or proprioception impairment

Numerous types and styles of electrode implants (for example, implants including one or more electrodes for providing an electrical stimulus) are available. Any implant for stimulation of sensory neurons in a subject may be utilized in specific implementations. In some implementations, more than one electrode is implanted, such as an array of electrodes. In additional implementations, a device is provided that can include one or more electrodes. The one or more electrodes are typically contained within an array of electrodes, such as an array of independently controllable electrodes on a percutaneous lead or an array of independently controllable electrodes on a paddle lead. Additional non-limiting examples include penetrating microarrays (e.g., Utah and Michigan microarrays) and microwire electrodes and arrays. Non-limiting examples of paddle arrays and their use are provided, for example, in US2009/0351221 and US2019/0366077.

Any neurostimulator suitable for stimulating the dorsal roots or dorsal rootlets of one or more sensory neurons innervating a limb of a subject and/or DRG can be used in the method provided herein. The neurostimulator includes a device for generating electrical current (the stimulator) that is connected to the one or more electrodes implanted in the subject. Typically, the stimulator is suitably designed for application of various current, voltage, pulse rate, waveforms etc. In some implementations, the neurostimulator is a pulse generator. In several implementations, the neurostimulator is a commercially available FDA approved stimulator. A complex device with a higher density of electrode contacts and shapes and sizes that better conform to the anatomical target may also be implanted to function as a stimulator.

The neurostimulator includes pulse generation circuitry that provides electrical conditioning and stimulation energy in the form of a pulsed electrical waveform to the implanted electrode array in accordance with a set of stimulation parameters programmed into the neurostimulator. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, including the pulse amplitude, pulse duration, pulse rate, and burst rate, etc.

Electrical stimulation will occur between two (or more) activated electrodes, one of which may be local to the neurostimulator or a part of the body distal to the spinal cord (e.g. an external patch electrode on the hip). Simulation energy may be transmitted to the tissue in a monopolar or multipolar (e.g., bipolar, tripolar, etc.) fashion. The stimulation energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative.

Non-limiting examples of controllable neurostimulation systems are provided in US2020/0254260, US2020/0360693, US2020/0360697, US2020/0152078, U.S. Pat. Nos. 10,252,065, 10,799,702, US2021/0016093, and US2020/0391030, each of which is incorporated by reference herein. Further, non-limiting examples of closed-loop neurostimulation systems are provided in U.S. Pat. Nos. 10,265,525, 10,279,167, 10,279,177, 10,391,309, 10,751,539, US. Pat. No. 10,981,004, and US2020/0147382, each of which are incorporated by reference herein.

In several implementations, the neurostimulator includes integrated circuitry to control the functions of the neurostimulator, including generation and application of electrical signals (via one or more channels of the electrodes implanted in the subject) to apply the electrical stimulus at the target location in the subject. The integrated circuitry can comprise and/or be included within a controller (e.g., processor) for controlling the operations of the neurostimulator, including stimulating, signal transmission, charging and/or using energy from a battery for powering the various components of the device, and the like. Typically, the neurostimulator includes a pulse generator that provides stimulation energy in programmable patterns adapted for direct stimulation of neuronal tissue.

The operable linkage of the neurostimulator to the electrode(s) can be by way of one or more leads, although any operable linkage capable of transmitting a stimulation signal from the neurostimulator to the electrodes may be used in specific implementations.

Post-operative control of selective electrical stimulation by the implanted electrode is provided in some implementations by a neurostimulator that may be externalized or implanted; for example, subcutaneously (e.g., in the chest or belly of the subject). Following recovery from the implantation, surgery, and connection of electrode leads to the neurostimulator, the subject may be monitored and tested to establish parameters for the electrical stimulation based on the subject's motor or proprioception impairment; for example, by monitoring one or more motor output(s) that provide a measurement of the extent of the impairment and the subject's response to stimulation. In some implementations, electrical stimulation by the implanted electrode(s) is delivered to sensory neurons in the subject while the subject performs a voluntary activity or task affected by the subject's impairment; for example, forelimb tasks (e.g., reaching, grabbing, picking with opposable thumbs, grip squeezing, and fine motor tasks involving precision finger movements) or lower limb tasks (e.g., walking, jumping, leg extensions).

In some implementations, the parameters of the electrical stimulus controlled by the neurostimulator are adjusted according to changes in the one or more motor or proprioception output(s) that are monitored while the subject performs a specific task, for example, so as to improve the motor outputs, thereby treating the subject's motor impairment. In particular implementations, the adjusted neurostimulator is part of a daily assistive device to treat the subject over an extended period of time. In specific implementations, the operation of the device and/or the neurostimulator can be at least partially under the control of the subject once the subject is released from a clinical setting. In these and further implementations, the subject is taught how to use the device and/or the neurostimulator.

The implanted electrodes and neurostimulator may remain in place for any suitable time period (such as about one month, about two months, about three months, about six months, about one year, or longer). In some implementations, the electrodes remain implanted in the subject for the duration of time that the method provides a therapeutic benefit to the subject.

In particular implementations, methods disclosed herein can be used in combination with protocolled physical rehabilitation exercises to improve long-term outcomes.

### V. Exemplary Implementations

Clause 1. A method for treating motor impairment and/or proprioception impairment due to neurological disorder or injury in a limb of a subject, comprising:
providing a therapeutically effective amount of stimulation to dorsal rootlets, lateral spinal cord adjacent to the dorsal rootlets, or dorsal root ganglia, of one or more sensory neurons innervating a limb of the subject with the motor impairment and/or proprioception impairment; and wherein:
the stimulation is provided with one or more electrodes of a neurostimulator that are implanted at the dorsal rootlets, lateral spinal cord adjacent to the dorsal rootlets, or dorsal root ganglia of the one or more sensory neurons innervating the limb of the subject;
the one or more electrodes are activated to provide the stimulation; and
the stimulation reduces the motor impairment and/or proprioception impairment in the limb of the subject.

Clause 2. The method of clause 1, wherein the motor impairment comprises loss of muscle strength, partial paralysis (such as paresis of the limb), loss of dexterity (such as hand finger movement), and/or uncontrollable muscle tone.

Clause 3. The method of clause 2, wherein the motor impairment comprises spasticity, aberrant flexor synergy, or contractures.

Clause 4. The method of clause 1, wherein the proprioceptive impairment comprises deficits in the ability to detect force generated by the limb and/or applied to the limb.

Clause 5. The method of clause 1, wherein the proprioceptive impairment comprises deficits in the ability to understand position of limb in space.

Clause 6. The method of any one of the prior clause, wherein the motor impairment and/or proprioception impairment is due to stroke.

Clause 7. The method of any one of the prior clauses, wherein the motor impairment and/or proprioception impairment is due to neuromuscular disorders such as Spinal Muscular Atrophy, Amyotrophic Lateral Sclerosis, Duchenne Motor Disorder.

Clause 8. The method of any one of the prior clauses, wherein the stimulation is applied at or below a motor threshold such that the stimulation does not directly elicit movement of the limb.

Clause 9. The method of any one of the prior clauses, wherein the limb is an arm of the subject and the neurostimulator is an epidural electrode array implanted at the dorsolateral aspect of the spinal cord and spanning the C3-T1 nerve roots.

Clause 10. The method of any one of the prior clauses, wherein the limb is a leg of the subject and the neurostimulator is an epidural electrode array implanted at the dorsolateral aspect of the spinal cord and spanning the T11-S1 nerve roots.

Clause 11. The method of any one of the prior clauses, wherein the neurostimulator is an external or implanted pulse generator.

Clause 12. The method of any one of the prior clauses, further comprising implanting the neurostimulator in the subject.

Clause 13. The method of any one of the prior clauses, further comprising selecting the subject with the motor impairment and/or proprioception impairment for treatment.

Clause 14. The method of any one of the prior clauses, wherein providing the therapeutically effective amount of stimulation comprises applying electrical stimulation at a frequency of from 1-130 Hz, an amplitude of from 1-12 mA, and a pulse duration of from 50-500 usec, to the dorsal rootlets, lateral spinal cord adjacent to the dorsal rootlets, or dorsal root ganglia innervating the limb of the subject.

Clause 15. The method of any one of the prior clauses, wherein the neurostimulator is implanted at the dorsal rootlets of the sensory neurons innervating the limb of the subject.

Clause 16. The method of any one of the prior clauses, wherein the neurostimulator is implanted at the lateral spinal cord adjacent to the dorsal rootlets of the sensory neurons innervating the limb of the subject.

Clause 17. An apparatus or system for treating motor impairment and/or proprioception impairment due to neurological disorder or injury in a limb of a subject as described herein.

Clause 18. An apparatus or system for performing the method of treating motor impairment and/or proprioception impairment due to neurological disorder or injury in a limb of a subject of any one of clauses 1-16.

### EXAMPLES

The following examples are provided to illustrate particular features of certain implementations, but the scope of the claims should not be limited to those features exemplified.

Globally, 1 in 4 people will suffer from a stroke. Of these people, nearly three quarters will exhibit lasting deficits in motor control of their arm and hand leading to enormous personal and societal impacts. These lasting deficits are partly due to the inability of current standards of care, physical and occupational therapy in the acute phase, to significantly reduce upper limb impairment.

Patients with chronic stroke exhibit a stereotypical motor syndrome of the upper limb that can be decomposed into independently quantifiable deficits: loss of strength, reduced dexterity, intrusion of aberrant synergies as well as a variety of muscle tone abnormalities such as hypertonia or spasticity. This paresis phenotype emerges from damage to the cortico-spinal tract (CST), which disrupts connections between the cortex and the cervical spinal circuits controlling arm and hand movements.

The examples provided herein show that engaging spinal circuits with targeted electrical stimulation immediately improved voluntary motor control in participants with chronic post-stroke upper limb paresis. A pair of multi-contact percutaneous epidural leads were implanted in the epidural space on the dorsolateral aspect of the cervical spinal cord to selectively target the C3-T1 dorsal roots. Stimulation obtained independent activation of shoulder, elbow and hand muscles. Continuous stimulation through selected contacts at specific frequencies enabled participants to perform movements that they had been unable to perform for many years. Overall, stimulation improved strength, kinematics, and functional performance. Unexpectedly, participants retained some of these improvements even without stimulation, suggesting that spinal cord stimulation could be a restorative as well as an assistive approach for upper limb recovery after stroke.

### Example 1

### Methods

### Trial and Subject Information

### Inclusion Criteria

Subjects between 21-70 years of age who had suffered from an ischemic or hemorrhagic stroke more than 6 months prior to the start of the study were eligible for participation. All subjects had hemiparesis affecting their upper limb and had a pre-study FM-UE score between 7 and 45. Prior to the study, participants were screened via a medical evaluation. Candidates with severe co-morbidities, previously implanted medical devices, claustrophobia, or who were pregnant, or breastfeeding were excluded from the study. Subjects were not receiving any anti-spasticity, anti-epileptic, or anti-coagulation medications for the duration of the study period.

### Study Design and Data Reported

This clinical trial provides evidence of safety and efficacy of Spinal Cord Stimulation (SCS) to improve motor control in people with chronic post-stroke upper-limb hemiparesis. The study was designed as a single-group, open-label, prospective study in participants with chronic stroke. Given the pilot nature of the study, to minimize safety risks, SCS leads were implanted for a maximum period of 29 days, after which the electrodes are explanted. Primary and secondary outcomes were designed to primarily assess safety and obtain preliminary clinical and scientific evidence of both assistive and long-term effects of SCS.

Briefly, after screening and pre-study baselines, subjects were implanted with clinical SCS leads. Starting from day 4 post-implant, subjects underwent scientific sessions 5 times per week, 4 hours per day, for a total of 19 sessions until explant day. Tasks and measurements during the first 5 to 7 sessions are focused on identifying optimal stimulation configurations that are then maintained for the remaining sessions.

Briefly, primary outcomes are focused on reporting serious adverse events and assessing pain/discomfort. Specifically, the trial is considered to be successful if there are no serious adverse events related to the use of the stimulation. Subjects are asked to report and rate, if present, any pain or discomfort that arises from the stimulation with the goal of understanding if intensities required for motor effects are pain/discomfort-free. Secondary outcomes are geared towards scientific and preliminary efficacy goals. In terms of clinical efficacy, immediate improvements in strength by measuring isometric forces with and without SCS are quantified one per week. Motor deficits are rated by assessing the Fugle-Meyer evaluation, ARAT assessment pre-study and on the last day of implant, and spasticity via Modified Ashworth Scale daily. Function is evaluated by measuring kinematics of the arm and hand during 2D and 3D reaching and grasping tasks. Finally, a battery of imaging and electrophysiology testing is performed to assess excitability and plasticity of spinal circuits pre-, during and post-study. The methods for each of the measurements reported in this trial that are part of the secondary outcome assessments is detailed below.

### Subject Information

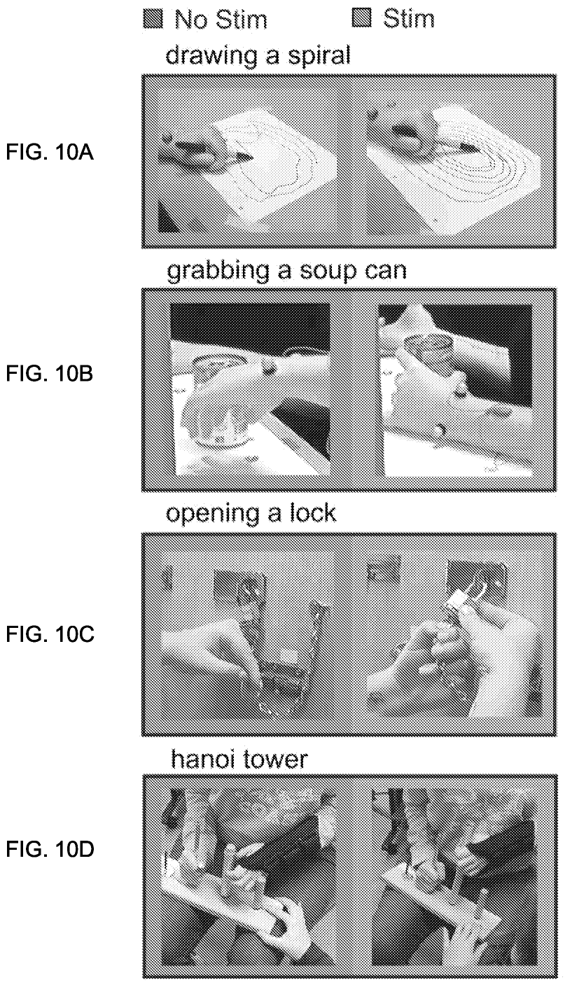

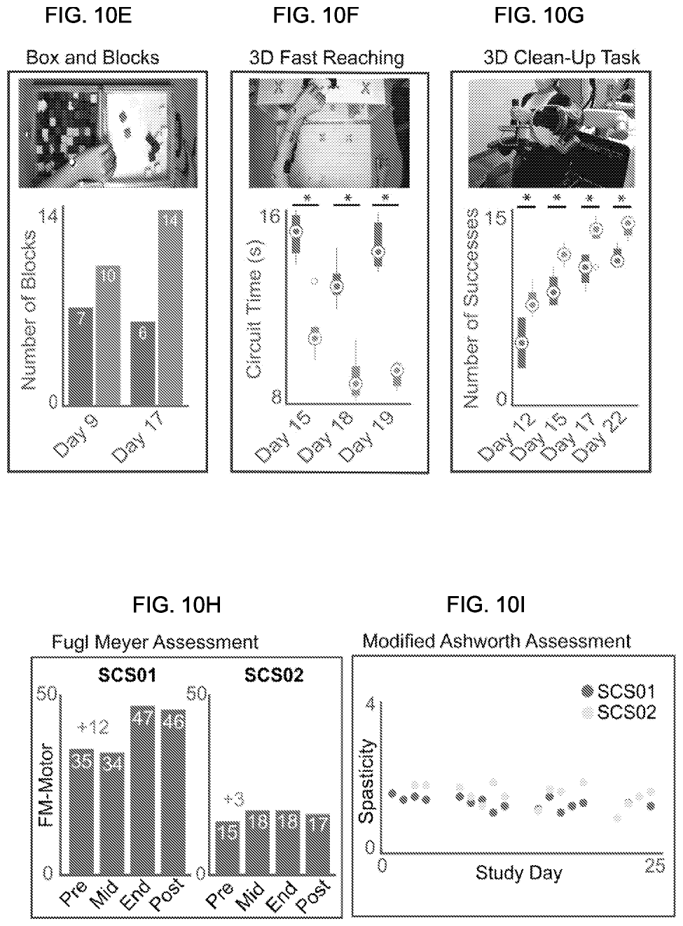

Results are reported herein from the first 2 subjects participating in the trial, both of whom were Caucasian females. SCS01 (31 years) had a right thalamic hemorrhagic stroke secondary to a cavernous malformation 9 years prior to participation in the study. Her interim history involved several bleeding events with eventual ablation of the malformation with gamma knife radiosurgery. At the time of her participation in the trial, her post stroke residual was a left-sided spastic hemiparesis for which she was receiving botulinum injections in her biceps, brachioradialis, and pronator teres. Botulinum treatments were suspended starting 6 months prior to the study period and continuing through the end of the study. In the years between her initial infarct and participation, she also underwent a C5-6 anterior cervical discectomy and fusion to treat cervical stenosis as well as a flexor tendon lengthening surgery due to spasticity and suffered arm and wrist fractures in her affected arm. For SCS01, analysis of 138 isometric force test repetitions at multiple joints (54 stim off and 84 stim on) and 36 planar reaches (18 with SCS and 18 without SCS) are reported. Also reported are the results of simulated activities of daily living and other motor tasks that were performed at least 1 session per week (see FIG. 10). Because of technical and subject availability reasons Transcranial Magnetic Stimulation (TMS) tests were not performed prior to the study to obtain Motor Evoked Potential (MEP) maps on SCS01.

SCS02 (47 years) had a right ischemic middle cerebral artery stroke secondary to a right carotid dissection resulting in a large MCA territory infarct 3 years prior to participation in the study. Her post stroke residual at the time of participation was a left-sided spastic hemiparesis complicated by a left wrist flexion contracture despite treatment with splinting. For SCS02, analysis of 42 isometric force tests repetitions at multiple joints (21 stim off and 21 stim on) and 57 planar reaches (38 with SCS and 19 without SCS) that were obtained across multiple days during the study are reported. Also reported are the results of simulated activities of daily living and other motor tasks that were performed at least 1 session per week (see FIG. 10). TMS measurements obtained over 9 locations and 11 muscles confirmed that SCS02 was MEP negative (e.g. no MEP present in any of the muscles of the paretic arm)

Both subjects successfully completed the protocol with no serious adverse events. SCS01 experienced phlebitis several days after the explant procedure at the end of the study that was resolved with oral antibiotics.

### Surgical Procedure

### Lead Implant and Explant

General anesthesia was induced using propofol and maintained using sevoflurane and propofol at levels that allowed for reliable somatosensory evoked potential monitoring. Short-acting paralytic was used for intubation, but no additional paralytic was given to facilitate intraoperative monitoring of SCS evoked EMG. Both subjects were placed prone and affixed in a 3-pin Mayfield head holder. The back and neck were prepared and draped in typical sterile fashion and prophylactic antibiotics were administered. A small incision was made over the T1-T2 laminas using fluoroscopic guidance, and the tissue was dissected to expose the fascia. A Tuohy needle was inserted into the T1-T2 epidural interspace and used to guide the placement of a clinically approved 8 contact percutaneous spinal lead (PN 977A260, Medtronic). The first (rostral) lead was threaded rostrally and steered in situ using fluoroscopy towards the lateral aspect of the spinal cord such that the most distal contact was positioned at the base of the C3 vertebral body.

To confirm placement of the distal lead and ensure that motor pools of the upper arm as proximal as the trapezius are recruited, current controlled monopolar stimulation was delivered using an intraoperative neuromonitoring system (Xltek Protektor, Natus Medical). Stimulation pulses were delivered at 1-2 Hz on representative electrodes of the array and compound muscle action potentials (CMAPs) were measured using intramuscular needle electrodes (ipsilateral trapezius, anterior deltoid, medial deltoid, posterior deltoid, biceps, triceps, pronator teres, wrist flexors, wrist extensors, abductor pollicis, and abductor digiti minimi; and contralateral bicep and wrist extensors). Contralateral activity was also recorded to ensure that SCS did not induce cross-over effects to the other arm. Once satisfied with the lead placement, the Tuohy needle was removed, and the lead was sutured to the fascia to prevent lead migration.

The second (caudal) lead was placed through the same incision and T1-T2 interspace, this time, ensuring that the most proximal contact was positioned at the T1 vertebral body. As before, intraoperative electrophysiology was performed to ensure proper placement, verifying that SCS could recruit motor pools of the most distal muscles in the hand including abductor pollicis and abductor digiti minimi. Once in final position, the two leads (rostral and caudal) overlapped to provide complete coverage of spinal segments C4 to T1. The distal ends of both leads were tunneled subcutaneously and exited through a separate stab incision over the left flank. Both incisions were closed, and the externalized portion of the leads were covered.

To explant the arrays at the conclusion of the study period, the patients were prepared in a similar fashion to the implantation surgery. The upper thoracic incision was re-opened, and the lead wires were cut and removed proximally. The distal end of the leads were removed through the lateral exit wound and both incisions were closed.

### Recruitment Curves

To evaluate the specificity of SCS in recruiting individual motor pools, recruitment curves were performed on each of the 16 contacts. Stimulation was delivered at 1-2 Hz on one electrode at a time with gradually increasing current amplitude while simultaneously recording CMAPs from all muscles. The peak-to-peak amplitude of the SCS-induced CMAPs were measured, one for each stimulus amplitude, and normalized to the maximum amplitude recorded on that muscle across all measured trials.

### Frequency Dependent Suppression

To validate that stimulation was activating dorsal sensory afferent fibers and not directly recruiting ventral motor efferent fibers, the stimulation frequency dependent response of CMAP amplitudes across several representative electrodes was evaluated. Current amplitude was fixed at a level above the motor threshold (the amplitude above which CMAPs were reliably induced). Pulse frequency was then increased from 1-2 Hz up to 20 Hz and the relative, normalized, peak-to-peak amplitude of CMAP responses were compared.

### Medical Imaging

### X-Ray Imaging

X-ray images were acquired at weekly timepoints in both axial and sagittal views to ensure the stability of lead position.

### Lesion Segmentation

MRI was acquired using a 3-T Prisma System (Siemens) using a 64-channel head and neck coil. A T1-weighted structural image was captured using a magnetization-prepared rapid gradient echo (MPRAGE) sequence (TR=2300 ms; TE=2.9 ms; FoV=256×256 mm²; 192 slices, slice thickness=1.0 mm, in-plane resolution=1.0×1.0 mm). Lesion segmentation was performed manually for each slice of the sequence using the MRIcron image viewer (NITRC) and the resulting region of interest (ROI) was smoothed on all planes using a gaussian smoothing kernel with a full-width at half-maximum of 2 mm. MRIcro_GL (NITRC) was used to visualize and export the resulting segmented overlays.

### High Definition Fiber Tracking (HDFT)

The same 3-T MRI scanner was configured to use a diffusion spectrum imaging scheme to capture a total of 257 diffusion samples. The maximum b-value used was 4000 s/mm² and the in-plane resolution and slice thicknesses were 2 mm. The accuracy of b-table orientation was examined by comparing fiber orientations with those of a population-averaged template (Yeh et al. NeuroImage 178, 57-68, 2018).

The diffusion data were reconstructed in the MNI space using q-space diffeomorphic reconstruction (Yeh et al., NeuroImage 58, 91-99, 2011) to obtain the spin distribution function (Yeh et al., IEEE Trans. Med. Imaging 29, 1626-1635, 2010). A diffusion sampling length ratio of 1.25 was used. The output resolution in diffeomorphic reconstruction was 2 mm isotropic. The restricted diffusion was quantified using restricted diffusion imaging (Yeh et al., Magn. Reson. Med. 77, 603-612, 2017). The tensor metrics were calculated and a deterministic fiber tracking algorithm (Yeh et al., PLOS ONE 8, e80713, 2013) was used to reconstruct the cortico-spinal tract fibers. A tractography atlas (Yeh et al. Neurolmage 178, 57-68, 2018) was used to map left and right cortico-spinal tracts with a distance tolerance of 16 mm. For the fiber tracking, the following were used: an anisotropy threshold of 0.035, an angular threshold of 50 degrees, and a step size of 1 mm. Tracks with lengths shorter than 10 mm or longer than 200 mm were discarded. A total of 1,000,000 seeds were placed. Topology-informed pruning (Yeh et al. Neurotherapeutics 16, 52-58, 2019) was applied to thetudy tractography with 16 iterations to remove false connections. the mean fractional anisotropy (FA) values for left and right cortico-spinal tract were calculated and the percentage of asymmetry was computed using Stinear's formula:

$$Asymmetry = \frac{{FA}_{H} - {FA}_{L}}{{FA}_{H} + {FA}_{L}}$$

Where FA_{L} is the mean FA value of the CST in the lesioned hemisphere and FA_{H} is the mean FA value of CST in the intact hemisphere.

### Custom Stimulation Controller

During the trial, SCS was delivered using a clinical grade, single channel, current controlled stimulator (DS8R, Digitimer) and a high-current compliant 1-to-8 multiplexer (D188, Digitimer). Current could be delivered to any contact by connecting it to the multiplexer and selecting the associated output channel. A custom-built microcontroller-based (Arduino Due, Arduino) control unit set pulse timing, amplitude, and output channel for each stimulus. Pulse width, inter-pulse interval, and waveform shape were fixed by the stimulator which ensured proper charge balancing and safe operation. Each pulse was a cathodic-first, biphasic square waveform with 200 μs (SCS01) to 400 μs (SCS02) monophase pulse width and 10 μs inter-pulse interval. Cathodic and anodic phases were equivalent in amplitude and duration.

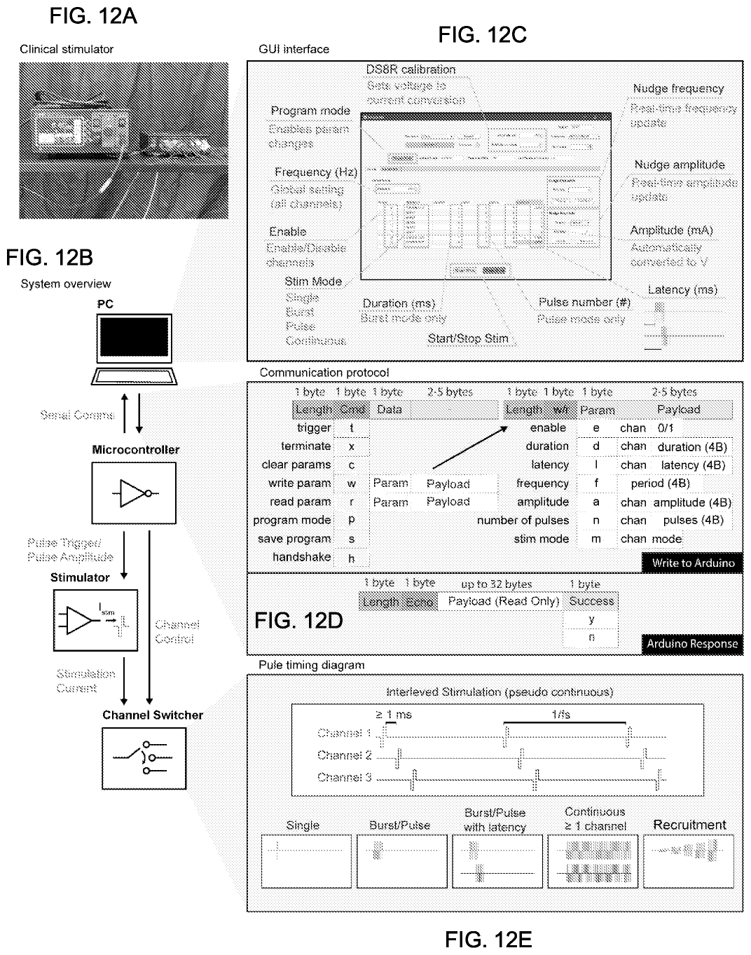

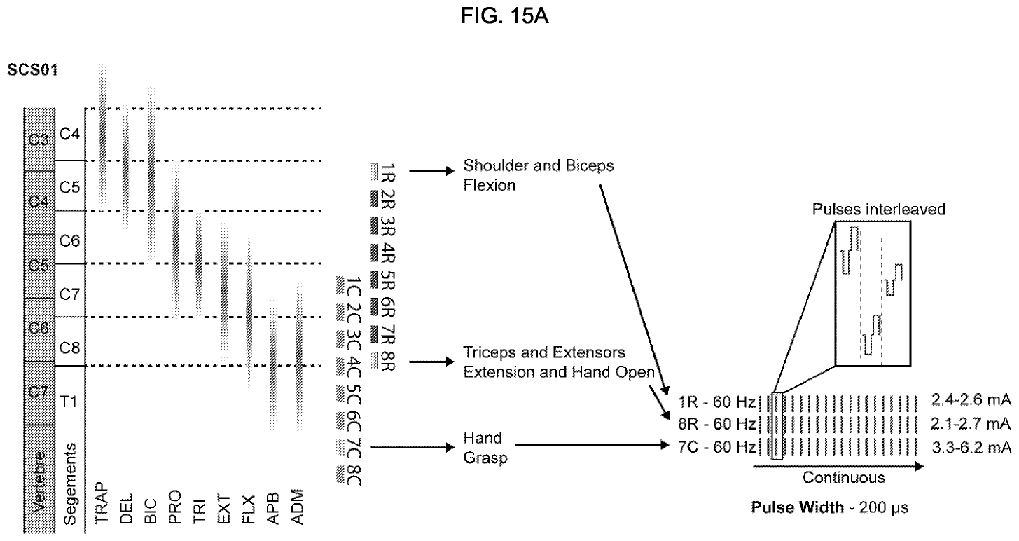

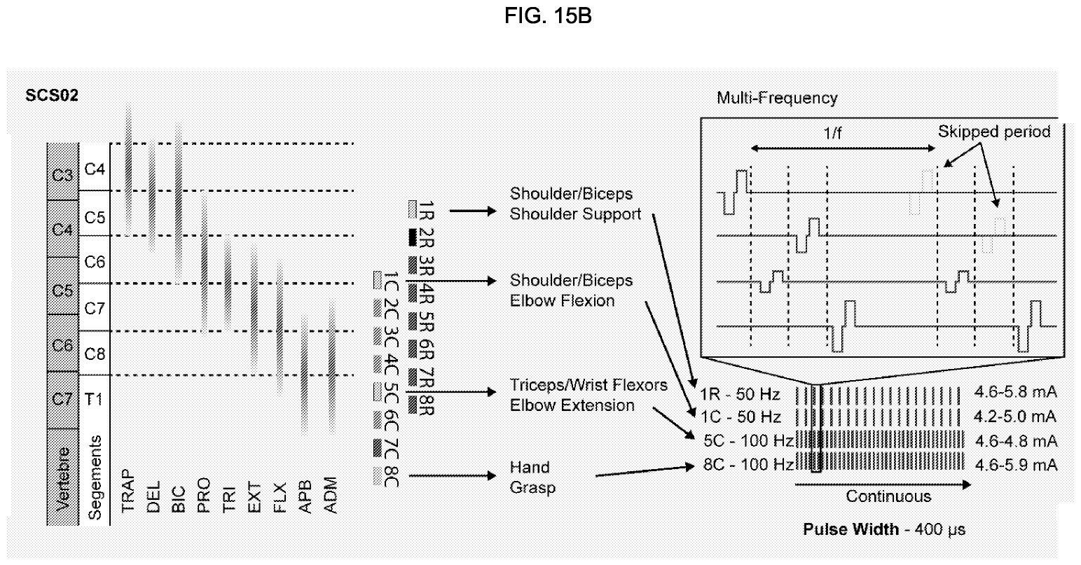

The control unit triggered each stimulus with a digital trigger pulse and set pulse amplitude using a continuous analog signal between 0-3.3 V. The DS8R hardware was configured for safety such that it could not produce amplitudes higher than 10.23 mA. Despite the stimulator comprising a single current source, the control unit's firmware enabled semi-synchronous stimulation across multiple channels by rapidly switching the output channel after each pulse (FIGS. 12E, 15). The time between pulses on separate channels was measured to be 2.2 ms, giving enough time for the multiplexer to fully switch output channels. During the study, this system was used to deliver stimulation on up to 4 separate spinal electrodes at up to 100 Hz. All programmable stimulation settings were configurable using a graphical user interface (GUI) developed in MATLAB which communicated with the control unit via a virtual serial port over a USB connection. Stimulation frequency, channel, duration, latency, and amplitude could all be configured manually via the GUI. Each channel could also be set to deliver a single pulse, a pulse train of fixed duration or pulse count, or continuous stimulation (FIG. 12).

A custom command protocol was implemented to facilitate communication between the GUI and control unit (FIGS. 12B, 12D). Communication was always initiated by the GUI with a command packet comprising the length in bytes of the packet, a 1-byte command, and 0-6 bytes of data. Possible commands included triggering or terminating stimulation, clearing the current configuration, reading or writing a parameter, configuring the microcontroller to accept new parameters (program mode), saving parameters, and an initialization handshake. When writing parameters, the length and command bytes were followed by the parameter to be set, the channel (if applicable), and the value to be written. When reading parameters, the data payload comprised only the parameter to be read. All commands were followed by a response packet from the microcontroller comprising the length of the packet, an echo of the command received, a data payload if applicable (for example when reading parameters), and a status byte indicating whether the command was executed correctly.

### EMG Acquisition

To assess muscle activity during movement, surface electromyography (sEMG) was recorded using a wireless EMG system (Trigno, Delsys Inc.). Up to 14 synchronized wireless sensors (Avanti Trigno, Delsys Inc.) were used to amplify, digitize, and wirelessly transmit EMG signals to a base station unit. Each sensor sampled the analog signal at 1925.925 Hz and applied a hardware bandpass filter of 20-800 Hz. Once the signals were received by the base station, they were converted back to an analog waveform and resampled at 2500 Hz by a data acquisition system (PCI-6255, National Instruments) for synchronization with other task events. The Trigno system has a known, fixed wireless latency of 59.6 ms.

At the beginning of each experimental session, the arm and hand were cleaned using isopropyl alcohol. Skin safe adhesive was used to secure the EMG sensors to the subject's arm. Depending on the muscles of interest for a particular experiment, from up to 14 individual muscles of the arm and hand were recorded; including the trapezius, anterior deltoid, medial deltoid, posterior deltoid, biceps, triceps, pronator teres, wrist flexors, wrist extensors, extensor digitorum, and abductor pollicis, whose locations were identified by palpation while the subject was instructed to perform simple movements. Sensors were then carefully removed at the end of each session.

### Efficacy Assessments: Single Joint Isometric Torque

Maximum isometric strength was measured for the shoulder, elbow, and wrist joints (when possible) using a robotic torque dynamometer (HUMAC® NORM™, CSMi). To measure torque, the robot's manipulandum was positioned and held at a fixed angle and the subject was asked to apply their maximum force while flexing or extending the joint under test for a sustained period of 5 seconds followed by a 10 to 15 second break. This procedure was repeated 5 times to complete a set. For each joint, the system was configured such that the joint was at a nominal and comfortable angle and so that it was aligned with the manipulandum's center of rotation. To isolate single joint function, the participants were constrained with tight straps at the shoulders as well as additional straps and bracing specific to each joint configuration of the HUMAC® NORM™. For example, while testing elbow strength, the upper arm and elbow were stabilized against the back of the chair while holding the manipulandum at a 90 degree angle. This ensured minimal shifting. Additionally, while testing wrist strength, the forearm was strapped to the robot's joint stabilization attachment. The HUMAC® NORM™ suggested configurations were used, when possible, but SCS02 was unable to support the weight of her arm and so was placed in a seated position to measure elbow and shoulder torques instead of the suggested supine position. In addition, a splint was used to secure SCS02's hand to the manipulandum to assist her in holding the handle securely and a counterweight was used where appropriate to offset the mass of the manipulandum and allow for more sensitive measurements. The maximum torque value within each repetition was considered for analysis.

Grip force was measured using a hand dynamometer. Participants were asked to hold the dynamometer and apply their maximum grasping force for five seconds. Before every repetition and after the participant's impaired hand was around the dynamometer, the device was zeroed out to ensure baseline grip force at rest was zero. Each measurement comprised the highest force produced on each of 3 attempts and data were combined across days to assemble enough data for statistical comparison.

### Efficacy Assessments: Clinical Impairment Scales

### Fugl-Meyer

The Fugl-Meyer Upper-Extremity assessment is a standardized evaluation of upper limb motor control and sensory function (Fugl-Meyer et al., Scand J Rehabil Med 7, 13-31, 1975). It includes 7 categories of assessments including passive and active range of motion, joint pain, proprioception, and tactile sensation. In total, there are 126 possible points. However, all scores reported herein correspond to the “Motor Function” sub-score which has a maximum value of 66. A trained medical professional conducted and scored the exam at 4 different timepoints: pre-study, mid-study (approximately 2 weeks after implant), end-of-study (4 weeks), and post-study (1 month after explant).

### ARAT

The Action Research Arm Test (ARAT) is another assessment of upper limb motor function that focuses on object interaction and manipulation. It comprises 4 categories including grasp, pinch, grip, and gross movement (Lyle. Int. J. Rehabil. Res. 4, 483-492, 1981). Scores can range from 0-57 with 57 representing the highest functional performance (Yozbatiran et al., Neurorehabil. Neural Repair 22, 78-90, 2008). A trained medical professional evaluated SCS01's ARAT performance both before the study, and at the end of the study. While SCS02's score was recorded at the pre-study timepoint, she did not consent to perform the test again at the end-of-study because of fatigue hence these data points are not available for SCS02.

### Modified Ashworth Scale

To ensure that SCS was not exacerbating joint spasticity, the Modified Ashworth Scale (MAS) was performed each session day at the beginning of the session. In order to minimize daily assessment duration, the joints tested for each participant were largely limited to those with spasticity prior to the study. However, elbow and digit flexion, shoulder external and internal rotation, and shoulder abduction were tested in both subjects, for consistency, regardless of prior history. This assessment involves passive manipulation of each joint, and ranking spasticity levels from 0-4 (0 being no spasticity). A trained medical professional performed and scored the assessment each day. Both a full breakdown of all joint scores measured on each day for both subjects as well as a “summary score” are reported. The summary score was taken to be the average score across all joints for each day.

### Functional Assessments: Planar Reach and Pull Kinematics

To evaluate upper limb motor control during directed reach and pull movements, a robotic augmented reality exoskeleton system (KINARM, BKIN Technologies) was used. Participants were secured in a modified wheelchair and their arms were suspended in the exoskeleton to remove the effects of gravity. The platform displayed virtual targets onto a dichroic augmented reality display in front of the subject that allowed them to visualize their hand position relative to the virtual graphics. The robot's motorized joints permitted the application of a mechanical load to the subject's movements.

### Center Out Task

For this task, the participants were asked to reach from a central starting position to one of 3 targets displayed using the AR display, then return to the starting position. On each trial the starting position was displayed, and the robot moved the subject's arm into position, locking it in place. Next the target was presented, and the exoskeleton was unlocked. An audio cue was played after a randomized 100 to 700 ms delay indicating that the subject could begin their movement. The participant was given 10 (SCS01) or 15 (SCS02) seconds to complete each trial. A target was considered acquired when the subject's index finger was within a 0.5 cm radius of the target center for 500 ms. An audio cue indicated the end of the reach phase. If the subject was unable to reach the target, the robot returned the arm to the starting position and the next target was presented. If the trial was successful, the subject's finger was positioned in the center of the target in preparation for the pull phase and locked in place. After a 500 ms delay, the arm was unlocked followed by a final audio cue after another 100-700 ms delay indicating the start of the pull phase, and the subject was required to return their hand to the starting position. In some trials, a load of

$$- 30 \frac{\text{?}}{m \text{?}}$$

$$\text{?} \text{indicates text missing or illegible when filed}$$

was applied isotropically to the movement using the exoskeleton to increase the task difficulty. Each target was presented 6 times in random order (unless otherwise noted). For each subject, appropriate targets were selected based on their individual range of motion.

The following metrics were calculated for each trajectory to compare kinematic quality. Trajectory smoothness was calculated as the number of peaks in the velocity profile for both the reach and pull phases. The total time of the combined reach and pull phases were also measured. Total path length was calculated and normalized to the Euclidean distance between the starting position and the target; more efficient movements had a lower value. Finally, the variance of each trajectory was calculated as the mean deviation of the actual trajectory from the mean trajectory calculated across all 5 repetitions of the movement.

### Open-Ended Reaching Task

The subject was presented with 3 equally spaced horizontal lines (approximately 15, 25, and 30 cm away from the participant) and was asked to reach from a starting position to the furthest line they could. In this way it was assessed how far the subject could reach in an open-ended manner. During each task, the participant started with their hand as close to their body as they could (maximum elbow flexion). After a verbal cue, they began their movement with the goal of passing the farthest line possible. Once the subject indicated that they had reached their maximum distance, another verbal cue indicated that they should return to their initial position. Task events were manually labeled during the trial by the experimenter. Each set comprised 5 repetitions.

As in the center-out task, a set of metrics was calculated for each trajectory; reach and pull phases were considered separately. Movement duration was calculated as the time it took from the beginning of each phase for the subject to cross the second horizontal line (25 cm) during reach and the first horizontal line during pull (15 cm). Maximum distance was measured as the axial distance between the point closest to the subject and the point furthest from the subject in each phase. Range of motion of the elbow during the task was considered as the angle difference between the most acute and most obtuse elbow angles achieved during each phase. As a metric of smoothness, the number of peaks in the elbow angle velocity profile was counted. Total path length measured the total length of the trajectory from the starting point to the second line (25 cm; reach phase) or from the end position to the first line (15 cm; pull phase) and was normalized by the phase duration. Finally, as a measure of variance, the distribution of each trajectory timepoint from the mean trajectory was calculated. A distribution skewed towards the left indicated that more samples were close to the mean trajectory, whereas a distribution with values towards the right indicated large deviations from the mean trajectory and therefore more variance.

### Functional Assessments: 3D Reaching

### Fast Reaching Task

The participant was presented with 6 targets, all axially equidistant from the subject, but at varying heights and lateral positions. The 3 “lower” targets were at table surface height and the 3 “upper” targets were raised to require shoulder flexion beyond 90 degrees. There was a left, center, and right target at each height. A 7^{th} position was placed directly in front of the subject and was used as a “home” position. Starting with their arm outside the working area, the subject was asked to first touch the home position then touch each of the 6 targets, returning to the home position after each target. For this task, the subject was asked to perform the sequence as fast as possible. The total time it took to reach all 6 targets was recorded.

### Robotic 3D Reaching Task

As an alternative to the fast-reaching task, an exoskeleton robot (ARMEO® POWER, Hocoma) was used to assist 3D movements when the subject was unable to lift their arm against the force of gravity (SCS02). This robotic system provides motorized support at each joint of the arm and measures kinematic variables in real time allowing for a subject's real-world movements to be displayed in a virtual video game environment. For this task, objects were presented within a virtual room and the subject was asked to reach toward each object and move it to a different position within the room (ARMEO® POWER cleanup game). The robot was configured to provide 50% weight support and assist movements at the “Low Support” setting. Game difficulty was set to “Easy”. Each game lasted 3 minutes and the goal was to move as many objects as possible within the time limit. The score was then recorded based on the number of objects successfully moved.

### Box and Blocks

When possible, the subject's performance in the “Box and Blocks” task was also evaluated. This is a standardized assessment in which a participant must grasp one small block at a time from one side of a box, lift it over a divider, and drop the block in the other half of the box. The total number of blocks moved from one side to the other within 1 minute was the subject's score.

### Functional Assessments: Activities of Daily Living

We chose ADLs after an initial assessment phase based on subject ability and preferences. In some instances, tasks were chosen that emulate everyday activities that participants had identified as being difficult to perform prior to the study; but that they wished to try after having experienced the stimulation. Since ADLs were customized for each participant, pre-study performance for these tasks was not evaluated.

### Drawing a Spiral

We asked the subject to draw a spiral shape using a marker on a plain piece of white printer paper taped down to a table. The goal of the task was to make the curves as smooth as possible and attempt not to overlap each of the concentric rings. The subject was allowed to comfortably position the pen in their hand using their unaffected hand before starting to draw.

### Object Manipulation

We placed a full, sealed can of soup on a table in front of the participant. The subject was asked to grasp the object from the side, requiring them to supinate their forearm, lift the can, and place it at an adjacent target. This task evaluated the subject's ability to reach, grasp, lift, and release a moderately heavy object. Here, the subject was not allowed to use their unaffected arm to assist in grasping the object.

In an alternative object manipulation task, the subject was asked to hold a wooden plank with vertical dowels (similar to a tower of Hanoi toy) on their lap using their unaffected hand. A metal cylinder was then placed over one of the dowels. The subject was required to grasp the cylinder, lift it off of the first dowel, align it and place it onto a second dowel, and release the cylinder. An experimenter helped to position the hand on the cylinder before the start of the trial. All other movements were performed by the subject entirely on their own.

### Lock Opening

As a measure of hand dexterity, a wooden panel with a shackle-style key-actuated lock was positioned on a table in front of the subject, who was asked simply to open the lock using their affected limb. To do this task, the participant was required to grasp and stabilize the lock with one hand (e.g. the unaffected hand), use a pinch grip to grasp the key with the other hand (e.g. the affected hand), and supinate the forearm to twist the key and unlock the lock. The subject then removed the lock from its latch on the wooden panel, replaced it by realigning the shank with the latch, and relocked the lock by aligning and pressing the shank back into the body.

### Self-Feeding

The subject was presented with small bite sized portions of food on a plate and a plastic fork. They were tasked with first picking up the fork from a table, using it to secure a piece of food, and perform the complex movement of orienting the food toward their mouth in preparation to eat it. Here, the subject was required to initiate picking up the fork with their affected hand but was allowed to reposition it using their unaffected hand before attempting to pick up the food.

### EMG Analysis

### Isometric Contraction (Root Mean Square Analysis)

During isometric contractions, EMG was acquired from appropriate muscles using wireless sensors as described above. Empirically, it was observed that deltoid EMG signals contained stimulation artifact during trials where stimulation was active due to the proximity of deltoid muscles to the stimulating electrodes. These artifacts were removed by blanking the signal coinciding with stimulation pulses. All signals were bandpass filtered (25-300 Hz, 5^{th} order Butterworth digital filter) and the root mean square (rms) value was calculated from the filtered data over the full duration of each trial for statistical analysis.

### Planar Reaching (Muscle Synergy Analysis)

Coordinated movements such as reaching and pulling require the timed co-activation of appropriate muscles to produce accurate and controlled limb motion. Muscles that were simultaneously active during planar reaching movements were measured by calculating muscle synergies using non-negative matrix factorization (NNMF), a dimensionality reduction technique (Israely et al., Front. Comput. Neurosci. 12, 2018).

EMG pre-processing was different for SCS01 and SCS02 due to large amplitude stimulation artifacts present in SCS02's EMG data that were not present for SCS01. For SCS01, stimulation artifact was removed by blanking and the resulting data were bandpass filtered (20-500 Hz, 5^{th} order Butterworth digital filter). For SCS02, EMG were first bandpass filtered using a narrower pass band (10-200 Hz, 5th order Butterworth, digital filter) to remove high frequency components of the stimulation artifact. Notch filters (5^{th} order Butterworth) at 50, 100, and 150 Hz were then used to remove low frequency harmonics of the stimulation artifact. The resulting signals from both subjects were rectified, low-pass filtered (5 Hz, 5^{th} order Butterworth digital filter), and normalized to the maximum EMG value recorded from that muscle over the whole day. Processed EMG was extracted from the reach and pull phases of each movement. Muscle synergies were identified using NNMF.

NNMF decomposes the EMG signals into a synergy activation matrix using the temporal correlation between the activity of individual muscles (Israely et al., Front. Comput. Neurosci. 12, 2018). The result is a set of one-dimensional timeseries signals for each muscle synergy identified. Each synergy in-turn comprises contributions from multiple muscles as described by a synergy vector. NNMF was implemented with two factors which were selected by observing the point-of-inflection in the residuals vs. number of synergies curve (Turpin et al., Eur. J. Appl. Physiol. 121, 1009-1025, 2021). For each phase of the movement (reach and pull), the primary synergy for that movement was identified as the one that most positively correlated (increased) with the movement. All repetitions of the movement were used to perform the dimensionality reduction. Finally, the contributions of deltoid and elbow muscles were quantified and compared using the primary synergy's synergy vector.

### Statistics

### Bootstrapping

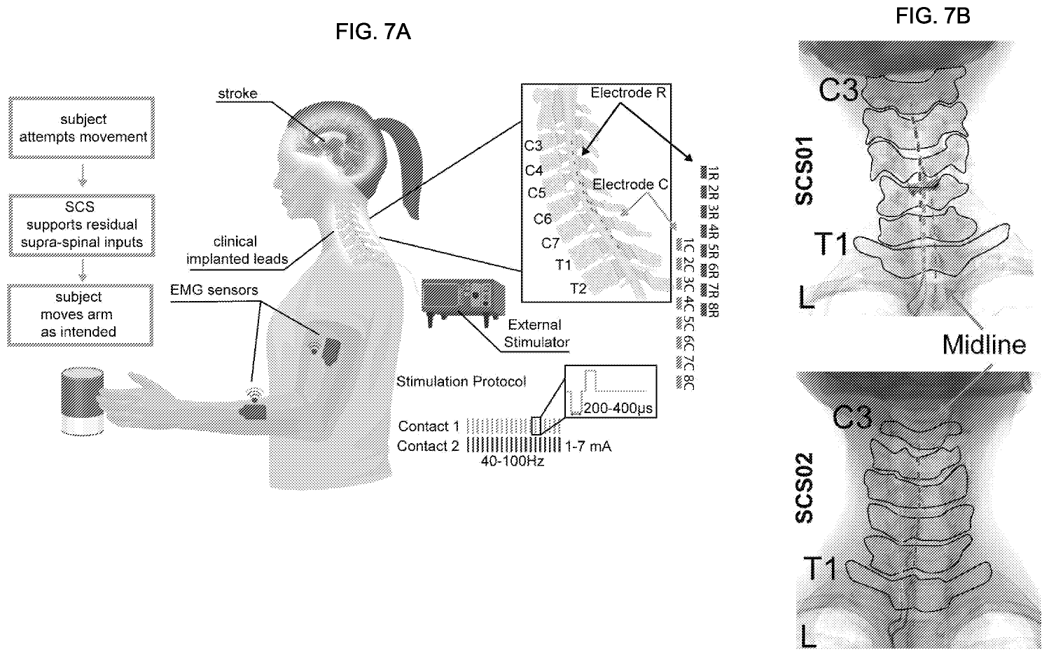

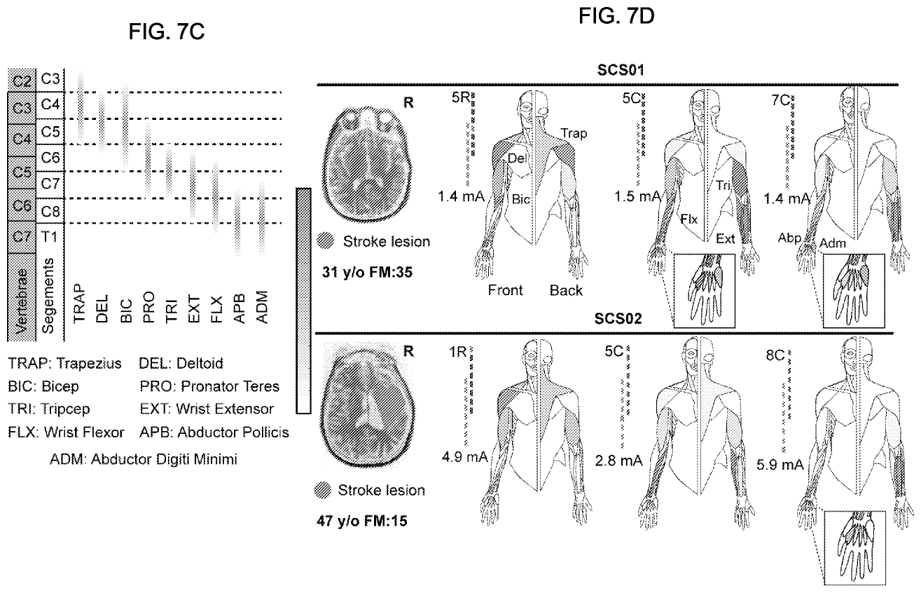

Statistical comparisons of means presented herein were performed using the bootstrap method, a non-parametric approach which makes no distributional assumptions on the observed data. Instead, bootstrapping uses resampling to construct empirical confidence intervals for quantities of interest. For each comparison (e.g. comparing stimulation on vs stimulation off for shoulder torque in SCS01, shown in FIG. 7F), bootstrap samples were construct by drawing a sample with replacement from observed measurements, while preserving the number of measurements in each condition. 10,000 bootstrap samples were constructed and, for each, the difference in means of the resampled data was calculated. A 95% confidence interval for the difference in means is obtained by identifying the 2.5^{th} and 97.5^{th} quantiles for the resulting values. The null hypothesis of no difference in the mean was rejected if 0 was not included in the 95% confidence interval. If more than one comparison was being performed at once, a Bonferroni correction was used by dividing this alpha value by the number of pairwise comparisons being performed.

### Comparison of Distributions

Statistical comparison of distributions was done using a two-sample Kolmogorov-Smirnov (KS) non-parametric test using MATLAB®. Again, an alpha value of 0.05 was used. Here, this test was used to compare the variability of kinematic trajectories during 2D planar reaching (the open-ended reaching task). The deviations of each trajectory from the mean trajectory were used to build a distribution of deviations. The resulting distributions for two conditions (stimulation off and stimulation on) could then be compared using the KS test.

### Example 2

### Experimental Framework to Quantify the Effects of SCS on Post-Stroke Motor Hemiparesis

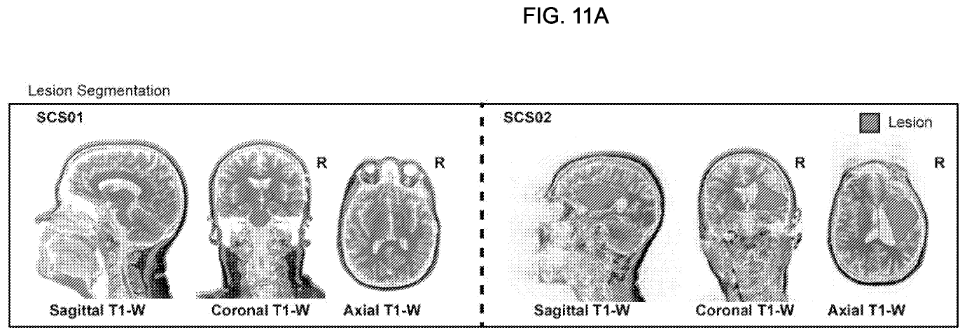

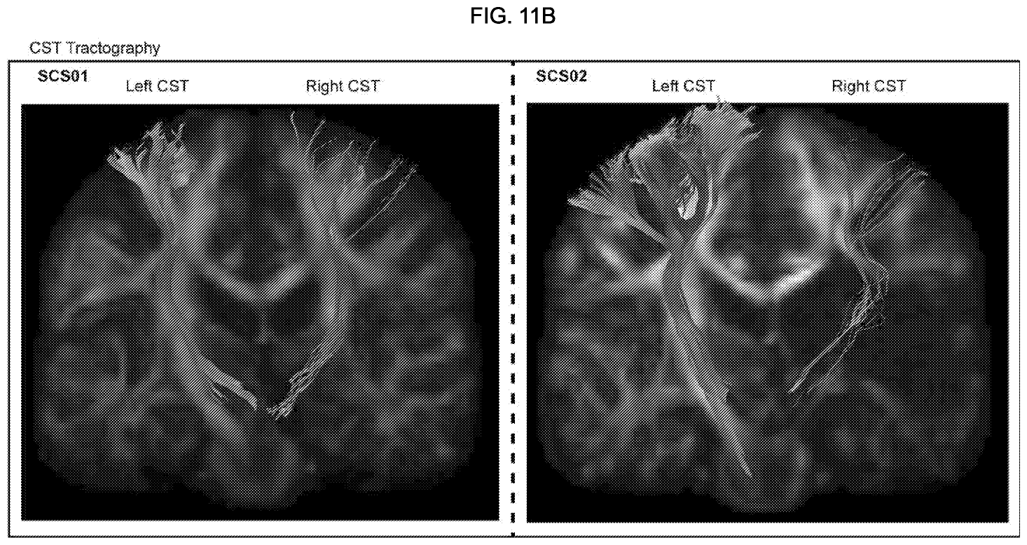

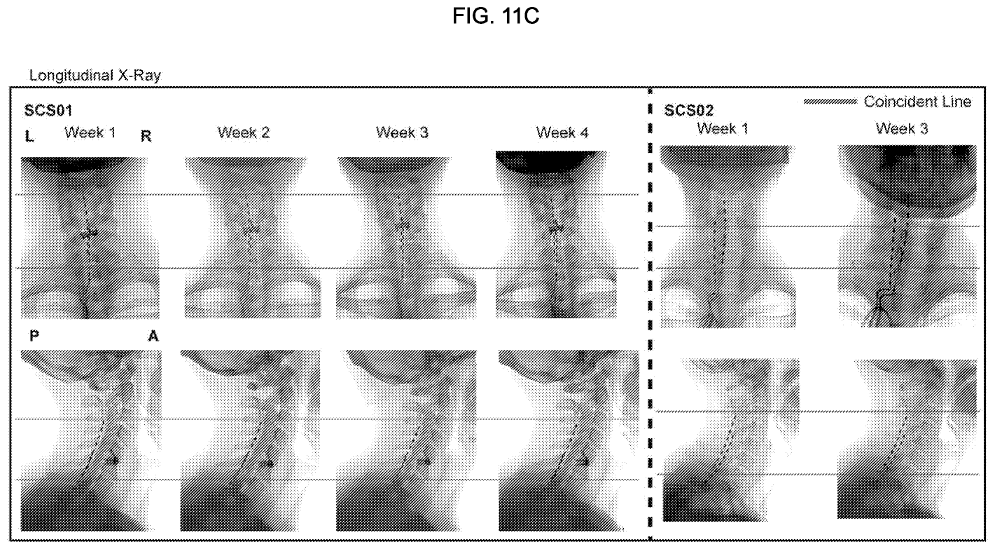

Two participants with chronic post-stroke upper limb motor deficits were recruited to evaluate the effects of continuous cervical SCS on motor performance (FIG. 7). SCS01 suffered an ischemic stroke while SCS02 suffered a hemorrhagic stroke 9 and 2 years prior to study enrollment, respectively. SCS01's lesion was localized to the internal capsule, midbrain, and pons, while SCS02's lesion was larger, affecting a large percentage of the corona radiata of the right hemisphere (FIG. 7D, FIG. 11). In both cases, extensive damage to the CST resulted in chronic upper limb impairment. A tractography analysis was performed using high-definition fiber tracking (HDFT) to compare the integrity of CST axons between the lesioned and healthy hemispheres of both participants (FIG. 11). Relative white matter integrity was measured by comparing Fractional anisotropy (FA) of the lesion hemisphere against the non-lesioned hemisphere and obtained FAS-0.17 for SCS01 and FAS=0.35 for SCS02 and (FAS=0 no impairment, see methods) indicating severe unilateral damage to the CST. This was reflected in pre-study Fugl-Meyer motor assessments of 35/66 (SCS01) and 15/66 (SCS02), indicative of moderate and severe impairment respectively.

This study was designed to quantify the immediate, assistive effects of continuous SCS on post-stroke motor deficits, including muscle weakness, impaired dexterity of arm and finger movements, intrusion of aberrant flexor synergies and tone disorders. However, when the immediate effects of SCS in SCI were present, combining SCS with physical training led to long-term therapeutic effects. Given the invasive surgical procedures for SCS lead implantation, it was reasoned that demonstrating immediate effects in a pilot trial is necessary before proceeding with follow-on studies to explore long-term effects of fully implanted SCS systems in stroke

Therefore, in this pilot trial, activity-based training exercises were not incorporated into the protocol and instead the trial focused on measuring immediate improvements attributable to the direct effects of SCS in facilitating motor function in the arm and hand. Testing began four days after implantation of the SCS leads and continued for four weeks, during which the subjects participated in assessments 5 sessions per week which each lasted approximately 4 hours. After 29 days the percutaneous leads were removed. Function with and without stimulation was evaluated that stimulation was delivered continuously through a custom-built microcontroller-based system connected via percutaneous access to the SCS leads participants (FIG. 12).

### Example 3

### SCS Achieved Segment-Level Specific Muscle Activation in the Cervical Spinal Cord

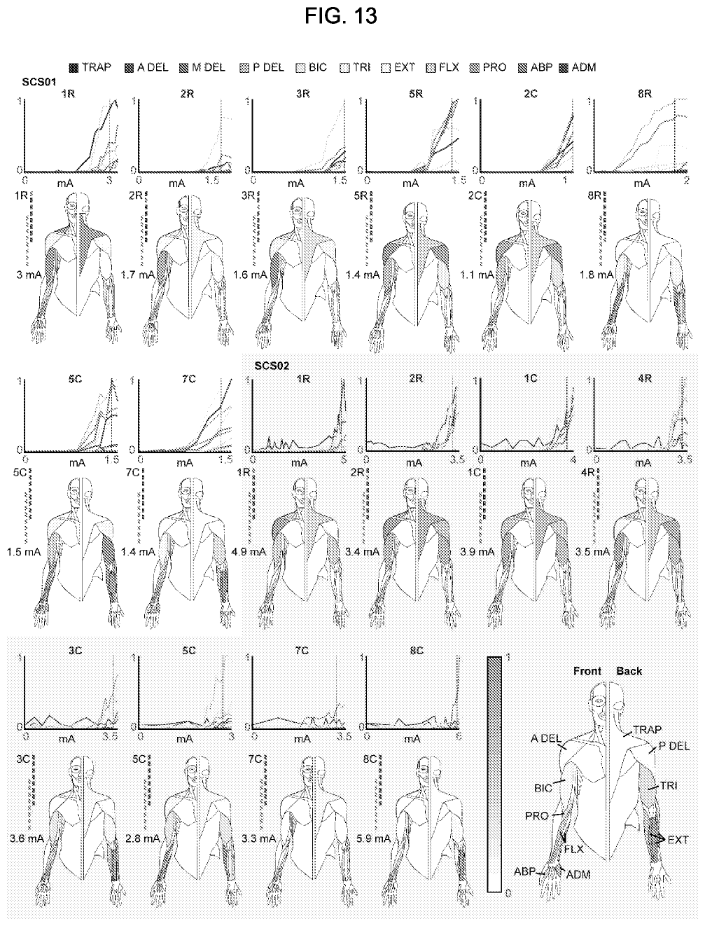

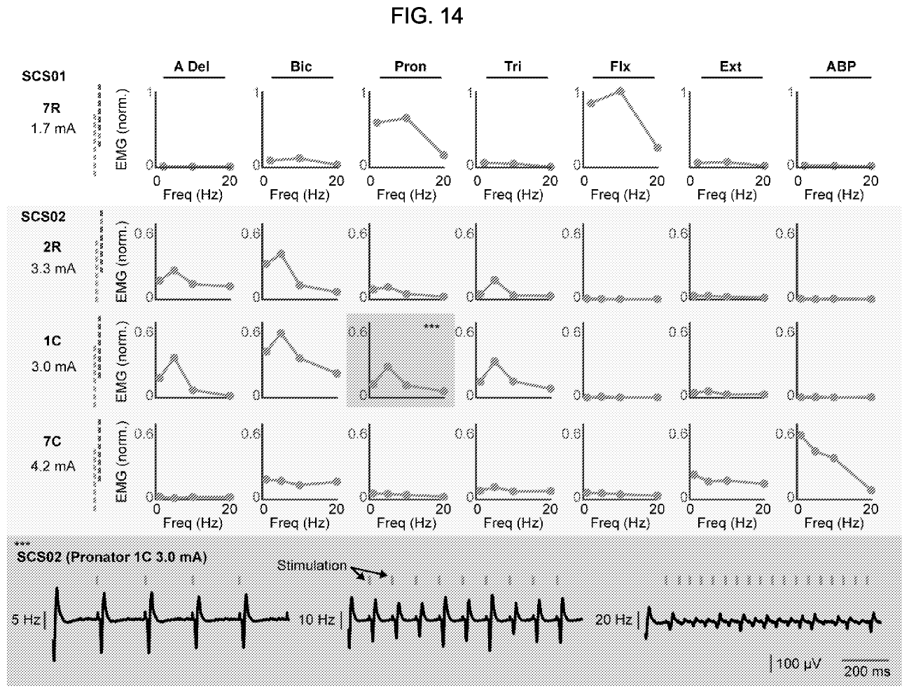

A surgical approach was designed to implant two linear electrodes mediolaterally spanning the dorsal roots C4 to T1 (FIG. 7B). During implantation, surgical placement was guided with neurophysiological intraoperative monitoring and it was verified that reflex-mediated muscle responses could be obtained reliably across all muscles of the arm and hand. Intra-operative data showed that SCS followed a clear rostro-caudal segmental specificity in both participants (FIG. 1D and FIG. 13). Monopolar stimulation of rostral contacts induced activity in the deltoids and trapezius while caudal contacts recruited intrinsic hand muscles (FIGS. 7F, 7H, 13). To verify that stimulation responses resulted from afferent-mediated recruitment of the motoneurons, and not by directly recruiting ventral roots, the same contact was stimulated at different frequencies (1.1, 2, 5, 10, and 20 Hz). Indeed, reflex mediated responses are well known to show frequency-dependent suppression phenomena. The peak-to-peak amplitude of evoked muscle activity was reduced significantly in a frequency dependent manner confirming that motor neuron activation was occurring trans-synaptically (FIG. 14). Repeated X-rays showed minimal rostro-caudal displacement of the leads from the implant (FIG. 11) which did not affect functional performances and the same contact configurations were used from week 2 (when they were finalized) to week 4 (FIG. 15). Stimulation intensity was adjusted daily to levels that enabled volitional movements but did not produce any passive joint movement or torques at rest.

In summary, it was demonstrated that accurate placement of clinical leads over the dorsolateral cervical spinal cord produces selective muscle activation according to well-described myotomal maps and that stimulation activates motor activity through sensory afferents in the dorsal roots.

### Example 4

### Arm and Hand Strength Immediately Improved Upon Activation of SCS

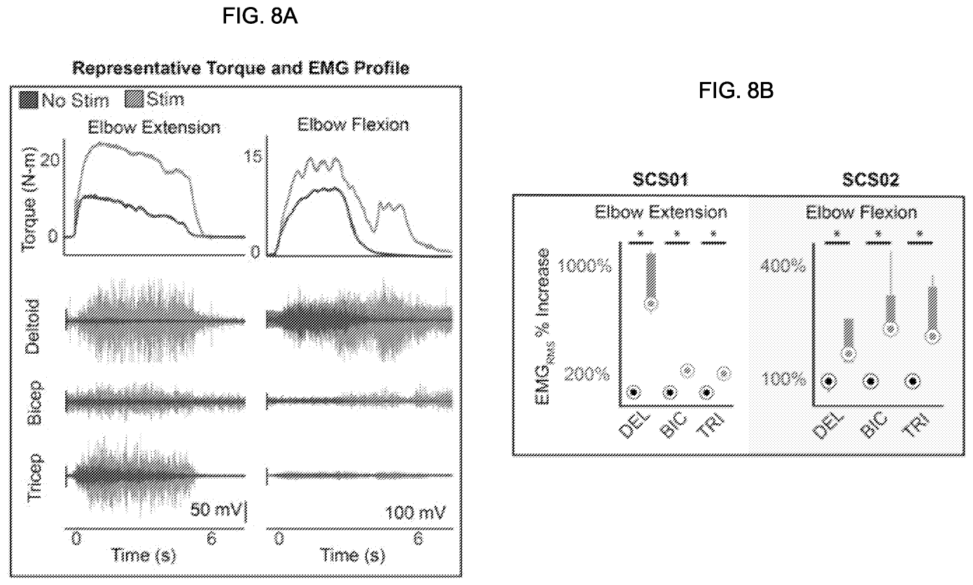

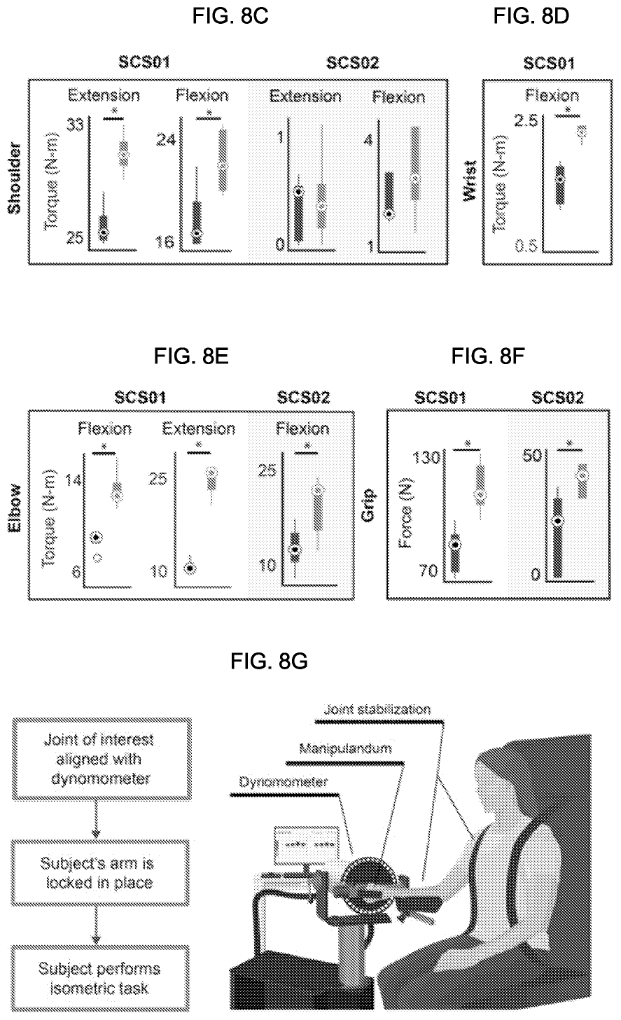

To determine whether SCS would lead to an increase in strength, participants were asked to apply their maximum force during isometric flexion and extension of single arm joints. Forces were applied to a robotic platform which measured joint torque (HUMAC® NORM™) (FIG. 8G). Torques produced with and without continuous SCS targeting muscles of the tested joint were compared (FIG. 2). SCS01 consistently increased strength for shoulder and elbow flexion and extension; mean torques at the elbow more than doubled when SCS was provided (Day 9:9.8 vs 22.0 Nm; Day 23:11.6 vs 24.6 Nm) (FIGS. 8A, 8D). At the wrist, SCS01 was unable to perform the wrist extension task with detectable forces even with SCS. However, consistent improvement in wrist flexion torques was measured (FIG. 8D). As an example of the functional relevance of these improvements, SCS01 could raise her arm above her head during SCS. In SCS01, multiple stimulation frequencies (20, 40 and 60 Hz) were tested during elbow flexion and extension isometric tests and it was found that 60 Hz yielded maximal torques. The severity of SCS02's impairment hindered consistent assessment of some joints. Specifically, she could produce detectable torques during shoulder flexion and extension and demonstrated significant improvements in elbow flexion torque (FIGS. 8A, 8C, 8E) similar to those observed in SCS01 (40% average increase), but elbow extension or wrist torques were not measurable either with or without SCS.

Additionally, isometric grip strength was tested using a hand-held dynamometer (FIG. 8F). SCS led to 40% increase in SCS01 and 108% increase in SCS02 suggesting that SCS can potentiate both arm and hand function. This result was particularly striking for SCS02 who had near complete left hand paralysis and was unable to consistently produce detectable hand grip forces (as measured with a hand dynamometer) without SCS. Additionally, on the first day of testing, SCS01, for the first time in the 9 years since her stroke, immediately reacquired the capacity to fully and volitionally open her hand. The root mean square values of EMG signals measured from the anterior deltoid, biceps, and triceps during elbow extension (SCS01) and elbow flexion (SCS02) were also compared. EMG was substantially higher with stimulation than without for these muscles in both participants (>100% increase, FIGS. 8B-8E) indicating that SCS potentiates the participant's ability to recruit muscles.

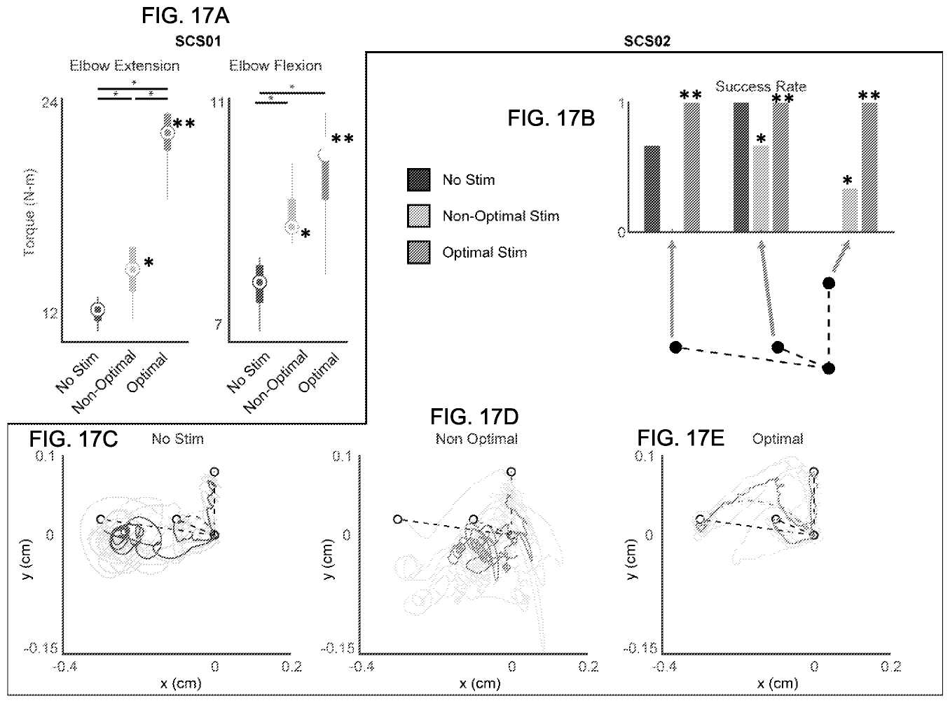

To test for the effects of motivation and bias, sham trials were performed in which non-optimal stimulation was delivered without participant knowledge. In these sham trials, electrodes were selected that preferentially activated muscle groups that were antagonistic to the movement performed (electrode 8R facilitated extension and 2R facilitated flexion). SCS01 still experienced paresthesia over the shoulder and arm during stimulation and was unable to distinguish optimal from sub-optimal configurations. While even antagonist stimulation led to some increase in strength (+19% extension using 2R, 2.2 mA, 60 Hz; 7C, 3.6 mA, 60 Hz and +16% flexion using 8R, 2.4 mA, 60 Hz; 7C, 3.6 mA, 60 Hz), the most significant improvements in strength occurred only when SCS was optimized for the intended movement (agonistic stimulation; +82% extension using 8R, 2.4 mA, 60 Hz; 7C, 3.6 mA, 60 Hz and +25% flexion using 2R, 2.2 mA, 60 Hz; 7C, 3.6 mA, 60 Hz) (FIG. 17). In summary, it was demonstrated that SCS led to immediate and substantial improvements in strength and muscle activity of the arm and hand when optimal contacts were used.

### Example 5

### SCS Improved Arm Motor Control During Planar Reaching

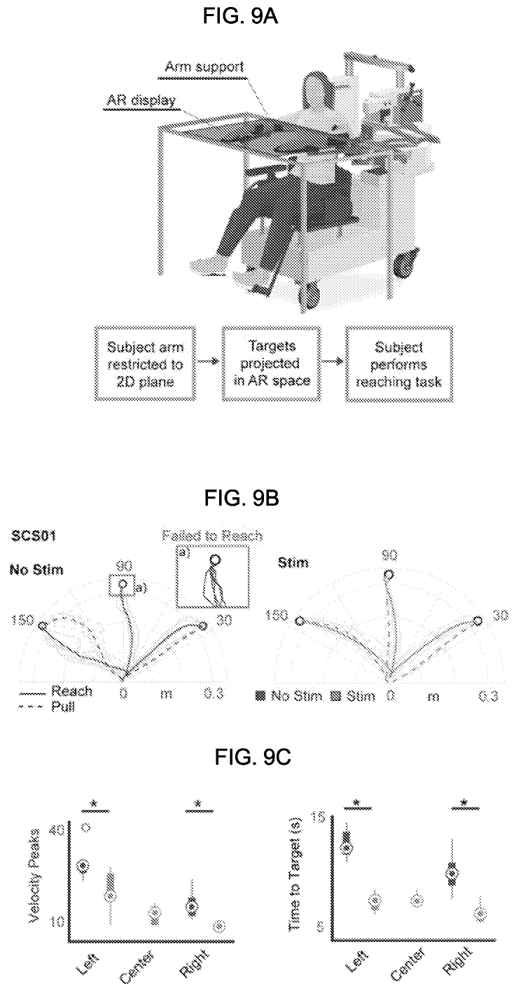

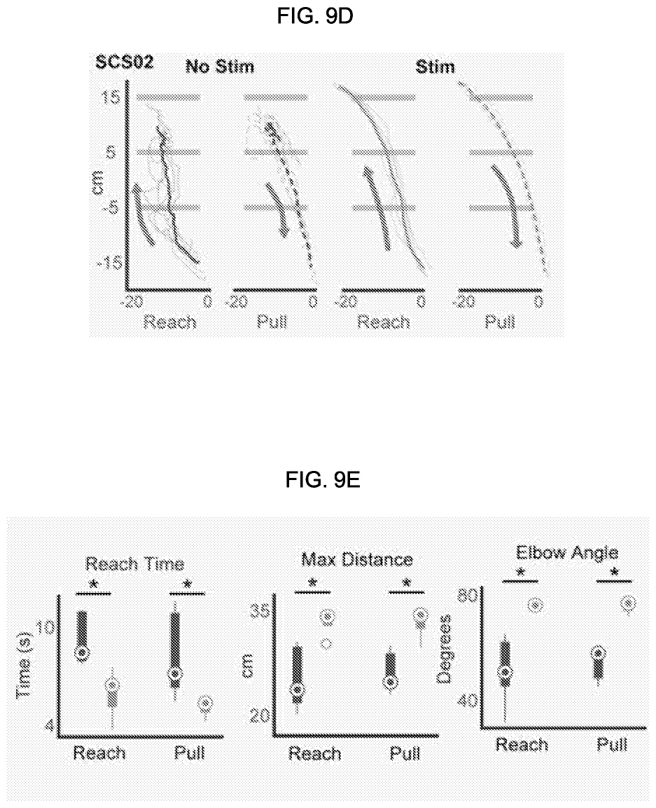

In addition to strength, the benefit of SCS on arm dexterity and muscle synergies was evaluated. For this, both participants performed planar reach and pull tasks using a robotic platform (KINARM®) that supported the weight of their arm (FIG. 9A). Importantly, these reaching tasks were performed in the horizontal plane to dissociate the effects of shoulder weakness and compensatory movements from the capacity of participants to extend their arm towards a target (Scott, J. Neurosci. Methods 89, 119-127, 1999).

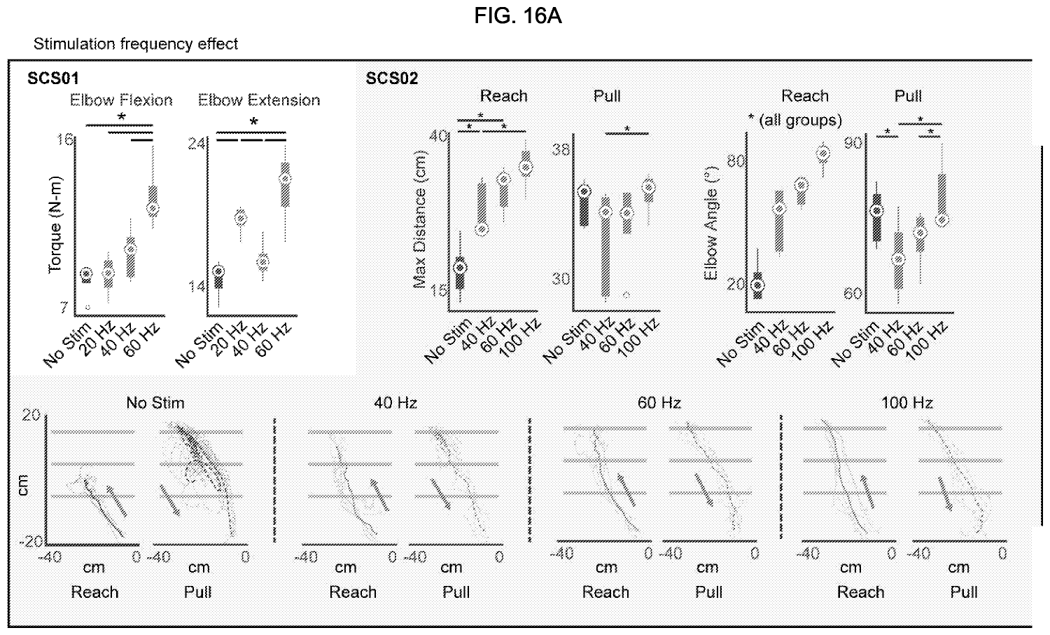

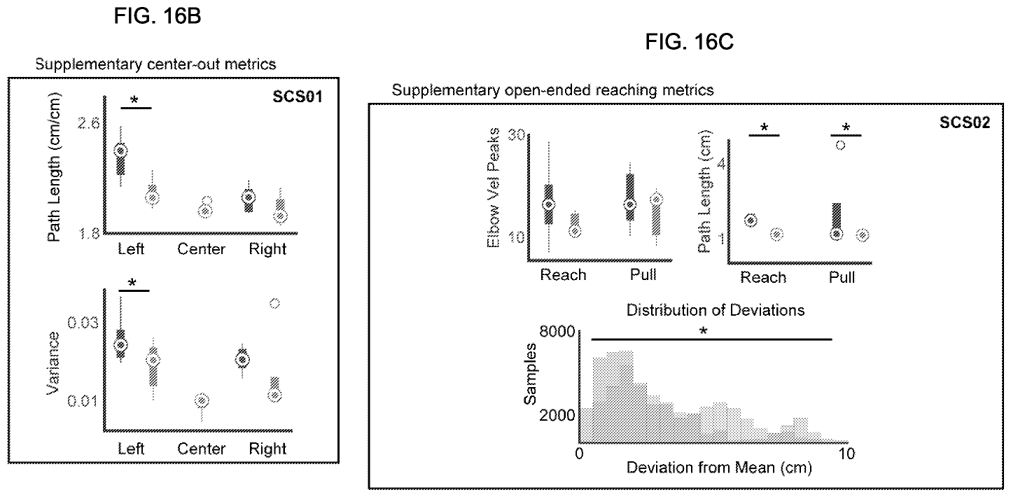

SCS01 was tasked with reaching towards different targets positioned to maximize active elbow extension since this was particularly difficult for the participant due to the intrusion of flexor synergies. During continuous stimulation, SCS01 was able to successfully reach all targets; whereas, without stimulation, she was never able to reach the central target because of difficulty extending her arm (FIG. 9B). Movements to targets that she could consistently reach with and without stimulation, were significantly faster and smoother with stimulation on (FIG. 9C; 34% (left target) and 47% (right target)). Similarly, speed (FIG. 9C) trajectory variability and max distance reached, were all improved with stimulation compared to controls (FIG. 16B). These results indicate that during stimulation SCS01 was able to perform smoother, faster, and more accurate movements as well as reach targets that she could not reach without stimulation.

Due to the severity of SCS02's impairment, to reduce frustration due to continued failure she performed a simpler task in which she was instructed to reach the furthest of three horizontal lines spaced at 10 cm intervals (FIG. 9D). Despite the simpler concept, the task assessed the same reaching and pulling arm kinematics in the same space as SCS01. Without stimulation, SCS02 was never able to reach the farthest line, but with stimulation on she was able to reach it on every trial due to the facilitation of elbow extension. This was reflected in the elbow excursion angle, which increased 23 degrees with stimulation (FIG. 9E). The maximum distance reached was 7.8 cm greater and total movement time was 37% faster with stimulation, FIG. 9E. Like SCS01, her movements also became smoother during stimulation (20% fewer velocity peaks FIG. 16C), and her trajectory variance and total path length also significantly improved (FIG. 16C). Arm extension kinematics and elbow angle were strongly modulated by stimulation frequency in SCS02 showing peak performances at 100 Hz (FIG. 16A).

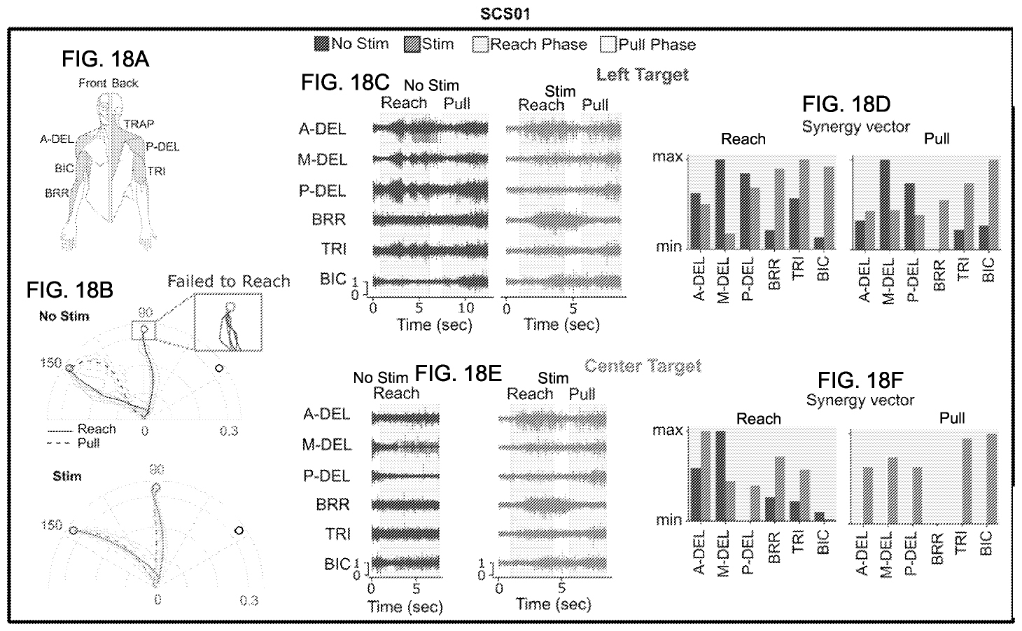

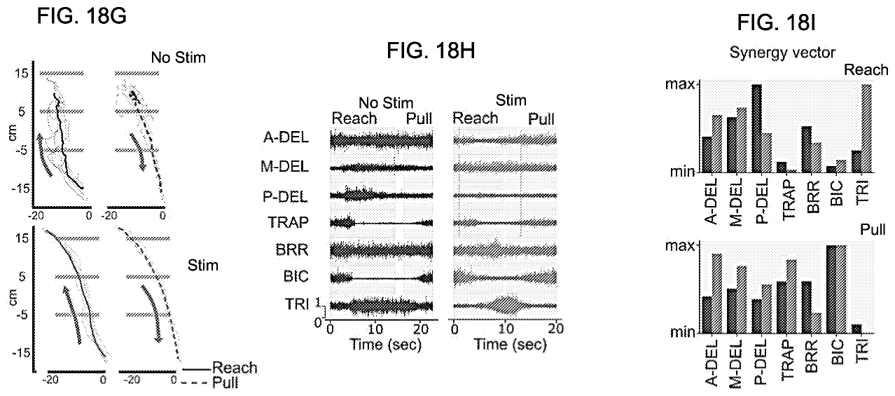

The improvements in reaching function were believed to be attributable to facilitation of elbow muscle activity and changes in flexion and extension synergies. To test this, EMG activities and extracted muscle flexor and extensor synergies associated with the extension and flexion movement phases were inspected using dimensionality reduction (FIG. 18, see methods). Without stimulation, muscle activity was very low at the elbow muscles and very high at the shoulder muscles, likely indicating a compensatory strategy dominated by shoulder muscles and allowing the elbow to extend passively during the reach. This was reflected in the strength of the components of each synergy that showed a greater contribution of shoulder muscles in both participants. Instead, with stimulation, the contribution of elbow muscles increased significantly and became dominant in both synergies which demonstrates facilitation of elbow muscle activity and changes in flexion and extension synergies, as well as a reduction of compensatory shoulder movements.

To test if stimulation specificity was necessary for optimal motor control, a sham-controlled task was performed in which frequency and amplitude matched non-optimal stimulation was delivered without participant knowledge in the center out task (sham: 4R, 4.4 mA, 50 Hz; 7R, 4.8 mA, 100 Hz; 8C, 4.6 mA, 50 Hz vs optimized: 1C, 4.4 mA, 50 Hz; 1R, 4.6 mA, 50 Hz; 5C, 4.8 mA, 100 Hz). FIG. 17 shows the dramatic impact of incorrect stimulation configuration on SCS02's task performance. Specifically, during sham-stimulation, arm kinematics suffered dramatically and her performance worsened, even compared to her ability with stimulation off, significantly affecting her ability to reach designated targets. Instead, with optimal stimulation she reached all targets 100% of the time.

In summary, despite differences in deficits and task difficulty, SCS targeting dorsal roots at specific cervical segments was shown to improved dexterity and enabled participants with stroke to perform smooth and effective arm movements enabling full elbow extension, improving elbow extension and flexion synergies and reducing compensatory shoulder activity.

### Example 6

### Functional Benefits of SCS

This example shows that improvements in strength and control observed with the SCS treatment translate to functional movements and improved performance during activities of daily living (ADL) (FIG. 10). Tasks were personalized for each subject according to impairment level and ADLs were selected based on observations of early-study improvements and the subjects' rehabilitation goals. First, the ability of SCS01 to perform 3D reaching movements was evaluated. SCS01 was asked to reach as fast as she could towards 6 targets placed on two different horizontal planes that required both planar and upward reaching movements against gravity. Continuous SCS enabled her to reach targets faster, approximately reducing in half the time required to complete the 6 target circuit (FIG. 10F). SCS01 was also asked to perform a classic manipulation task: the box and blocks task, in which she was instructed to move small cubic objects from one side of a box to the other by grasping and lifting them over a barrier. With stimulation on, she consistently performed better and, on day 17 post-implant, she more than doubled the number of blocks transferred when stimulation was off. Her score increased from 6 blocks without stimulation to 14 blocks during stimulation (FIG. 10E). Function was also assessed with the Action Research Arm Test (ARAT, Lyle, Int. J. Rehabil. Res. 4, 483-492, 1981). SCS01's pre-study baseline score was 31/57. At the end of the study, the test was administered both with and without stimulation, with resulting scores of 45/57 and 36/57 respectively; representing a 14 points improvement while SCS was active. Finally, the complexity of the tasks was increased by presenting activities of daily living that required high skill and dexterity such as drawing a spiral, reaching for and lifting a soup can, eating with a fork, and opening a lock. SCS increased her overall dexterity, allowing her to produce smoother and more consistent drawings (FIG. 10A). Stimulation also enabled simultaneous reaching, forearm supination and grasp allowing SCS01 to reach, grasp and lift a soup can. Without stimulation, forearm pronation and supination were not possible. This improvement of fine motor skills even enabled her to grasp a key and open a lock, whereas without stimulation she was unable to hold the key or lock at all (FIG. 10C). Finally, with stimulation, SCS01 was able to feed herself using her affected hand, with full independence; a task that she had been unable to perform for 9 years.

Since SCS02 was unable to sustain the weight of her arm against gravity, the 3D reaching task was adapted using a clinically-approved powered-exoskeleton robot (Hocoma ARMEO® Power) to provide titrated assistance during the task (50% arm weight support). It was endeavored to keep the movements as similar as possible to those performed by SCS01 to allow for comparison by having the subject collect virtual objects from a room and place them on a target (FIG. 10G). With stimulation, SCS02 was significantly more efficient at the task and managed to consistently reach towards more targets than without stimulation across three sessions (FIG. 10G). SCS02 was asked to perform a skilled motor task where she had to remove a hollow cylinder from a wooden dowel and slip it over another. With SCS she was not only able to grasp and lift the metal cylinder, but also to place it on the adjacent dowel without any weight support (FIG. 10D). Without SCS she could not complete any of the steps required for this task. These combined results show that the assistive effects of SCS can facilitate large improvements in functional performances and activities of daily living.

### Example 7

### Tone, Spasticity, Tolerability, and Unexpected Lasting Effects on Motor Control of SCS

To ensure that increased excitation to the spinal cord via SCS did not exacerbate spasticity or other muscle tone dysfunctions, the Modified Ashworth Scale was measured on each day of testing. To minimize daily assessment duration, joints tested were limited for each participant to those with MAS>1 prior to the study. However, elbow and digit flexion, shoulder external and internal rotation, and shoulder abduction were tested in both subjects, for consistency, regardless of prior history. Over the course of four weeks, it was found that SCS did not lead to any worsening nor amelioration in MAS scores (FIG. 101 and Table 2). In addition, the two participants did not report increased rigidity nor painful sensations during SCS. In fact, both patients described the stimulation as a “feeling of power in the arms” or a feeling of “being able to control my arm as if I know what I should do to move it”. Since this pilot was designed to study the assistive rather than the therapeutic effects of SCS, participants did not receive concomitant physical or occupational therapy over the four weeks. Thus, it was not expected to observe sustained improvements when SCS was turned off. Nevertheless, when the participants' pre- and post-study Fugl-Meyer scores were compared, SCS01 improved from 35 points at enrollment to 47 points, and SCS02 from 15 points to 18 points. These scores decreased by 1 point at a 4 week follow up visit. (FIG. 10H and Table 1). Finally, no serious adverse events were reported.

TABLE 1
Fugl-Meyer Assessment longitudinal breakdown. A breakdown table
of the scores for each of the 7 FM-UE assessment categories.
In bold, is the total score for the motor function subcategory
which is the sum of the Motor Upper Extremity, Motor Wrist, Motor
Hand, and Motor coordination/speed sections. The rightmost column
indicates the maximum possible score for each category.

| | | | | |
|---|---|---|---|---|
| | | | 1 mo. | |
| Pre- | Mid | End | Post | Total |
| Study | Study | Study | Study | Possible |

SCS01

| | | | | | |
|---|---|---|---|---|---|
| Passive Joint Motion | 21 | — | 21 | 21 | 24 |
| Joint Pain | 24 | — | 24 | 24 | 24 |
| Motor Upper extremity | 19 | 20 | 27 | 26 | 36 |
| Motor Wrist | 3 | 3 | 5 | 5 | 10 |
| Motor Hand | 9 | 7 | 11 | 11 | 14 |
| Motor | 4 | 4 | 4 | 4 | 6 |
| coordination/speed | | | | | |
| Total Motor Function | 35 | 34 | 47 | 46 | 66 |
| Sensation | 11 | 11 | 11 | 11 | 12 |

SCS02

| | | | | | |
|---|---|---|---|---|---|
| Passive Joint Motion | 22 | 20 | 20 | 20 | 24 |
| Joint Pain | 24 | 23 | 23 | 24 | 24 |
| Motor Upper extremity | 11 | 14 | 13 | 12 | 36 |
| Motor Wrist | 0 | 0 | 0 | 0 | 10 |
| Motor Hand | 0 | 0 | 1 | 1 | 14 |
| Motor | 4 | 4 | 4 | 4 | 6 |
| coordination/speed | | | | | |
| Total Motor Function | 15 | 18 | 18 | 17 | 66 |
| Sensation | 2 | 1 | 1 | 2 | 12 |

TABLE 2
Modified Ashworth Scale longitudinal breakdown. A breakdown
table of the individual MAS scores for each joint tested across
all days of the trial. In each case, a score of 0 corresponds
to no spasticity, and a score of 4 indicates no mobility at all.

| | | | | | | | | | |
|---|---|---|---|---|---|---|---|---|---|
| Study | Elb | Dig | Shld | Shld | Shld | FA | Wr | | Elb |
| Day | Flx | Flx | ER | IR | ABD | Sup | Flx | Pron | Ext |

SCS01

| | | | | | | | | | |
|---|---|---|---|---|---|---|---|---|---|
| 1 | 1.5 | 1.5 | 1.5 | 1.5 | 1.5 | 2 | — | — | — |
| 2 | 1.5 | 1 | 1.5 | 1.5 | 1.5 | 1.5 | — | — | — |
| 3 | 1.5 | 1.5 | 1.5 | 1.5 | 1.5 | 1.5 | — | — | — |
| 4 | 1 | 1 | 1.5 | 1.5 | 1.5 | 2 | — | — | — |
| 7 | 1.5 | 1.5 | 1.5 | 1.5 | 1.5 | 1.5 | — | — | — |
| 8 | 1 | 1 | 1.5 | 1.5 | 1 | 2 | — | — | — |
| 9 | 1 | 1.5 | 1.5 | 1.5 | 1 | 2 | — | — | — |
| 10 | 1 | 1 | 1 | 1 | 1 | 1.5 | — | — | — |
| 11 | 1 | 1 | 1.5 | 1 | 1 | 2 | — | — | — |
| 14 | 1 | 1 | 1 | 1 | 1.5 | 1.5 | — | — | — |
| 15 | 1.5 | 1.5 | 1.5 | 1.5 | 1 | 2 | — | — | — |
| 16 | 0 | 1 | 1.5 | 1.5 | 1 | 1.5 | — | — | — |
| 17 | 1 | 1.5 | 1.5 | 1 | 1 | 1.5 | — | — | — |
| 18 | 1 | 1.5 | 1 | 1 | 1.5 | 2 | — | — | — |
| 22 | 1 | 1 | 1.5 | 1.5 | 1.5 | 1.5 | — | — | — |
| 23 | 1 | 1.5 | 1.5 | 1.5 | 1.5 | 2 | — | — | — |
| 24 | 1 | 1.5 | 1 | 1 | 1 | 2 | — | — | — |
| Post | 1 | 1.5 | 1.5 | 1.5 | 1 | 1.5 | — | — | — |
| study 52 | | | | | | | | | |

SCS02

| | | | | | | | | | |
|---|---|---|---|---|---|---|---|---|---|
| 1 | 2 | 1.5 | 2 | 0 | — | — | 3 | 1.5 | — |
| 2 | 2 | 1.5 | 2 | 1.5 | 2 | — | 3 | 1.5 | — |
| 3 | 2 | 1.5 | 2 | 1 | 2 | — | 3 | 1.5 | 1.5 |
| 4 | 2 | 1.5 | 2 | 1.5 | 1.5 | — | 3 | 2 | 1 |
| 7 | 2 | 1.5 | 2 | 1 | 1.5 | — | 3 | 1.5 | 1.5 |
| 8 | 2 | 2 | 2 | 1.5 | 1.5 | — | 3 | 0 | 0 |
| 9 | 2 | 2 | 1.5 | 0 | 1.5 | — | 3 | 0 | 0 |
| 10 | 2 | 1.5 | 2 | 1.5 | 2 | — | 3 | 2 | 1 |
| 11 | 2 | 2 | 2 | 0 | 1 | — | 3 | 1 | 1 |
| 14 | 2 | 1 | 1.5 | 0 | 1.5 | — | 3 | 0 | 0 |
| 15 | 2 | 1.5 | 2 | 1.5 | 1.5 | — | 3 | 1 | 1 |
| 16 | 2 | 1.5 | 1.5 | 1 | 1.5 | — | 3 | 1.5 | 1 |
| 18 | 2 | 1.5 | 2 | 1.5 | 2 | — | 3 | 2 | 1 |
| 21 | 1.5 | 1 | 1 | 0 | 0 | — | 3 | 1 | 0 |
| 22 | 2 | 1 | 1.5 | 0 | 0 | — | 3 | 1 | 2 |
| 23 | 2 | 1.5 | 1.5 | 1 | 1.5 | — | 3 | 1.5 | 0 |
| 24 | 2 | 1.5 | 2 | 1 | 1 | — | 3 | 1.5 | 1 |

### Example 8

### SCS Improves Proprioception

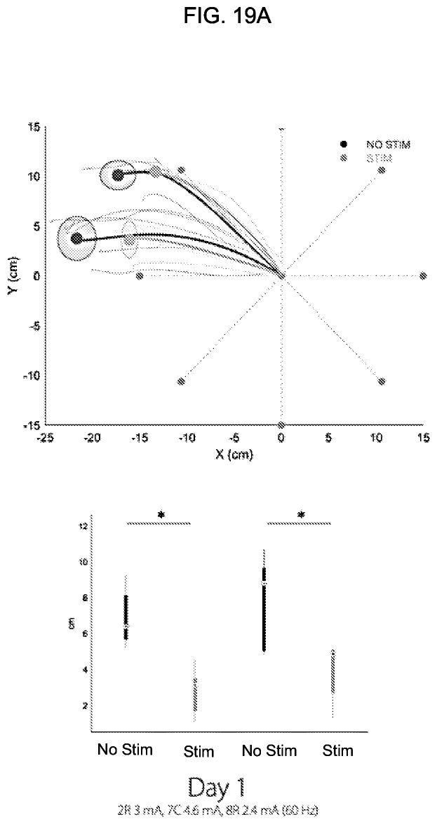

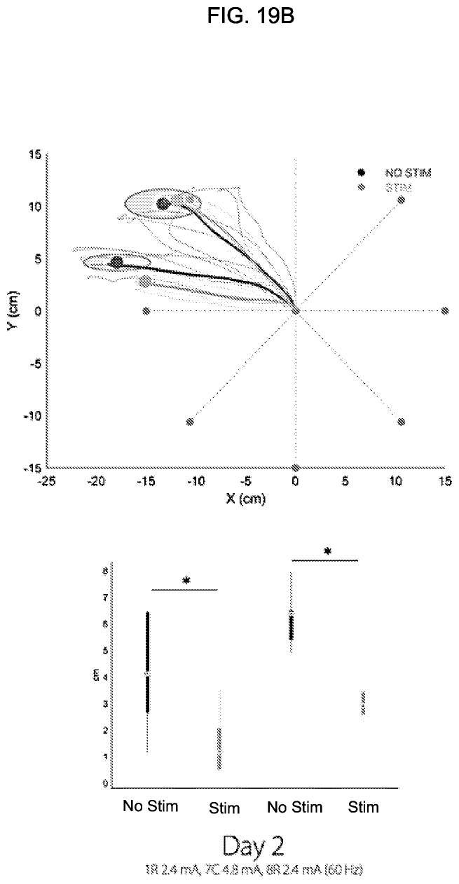

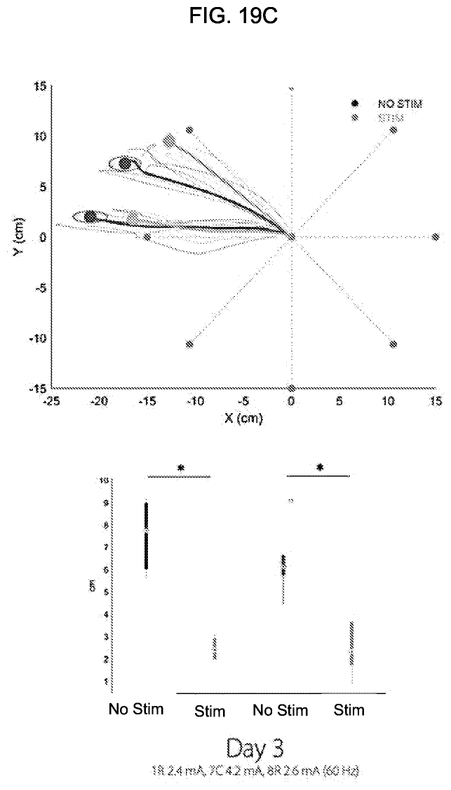

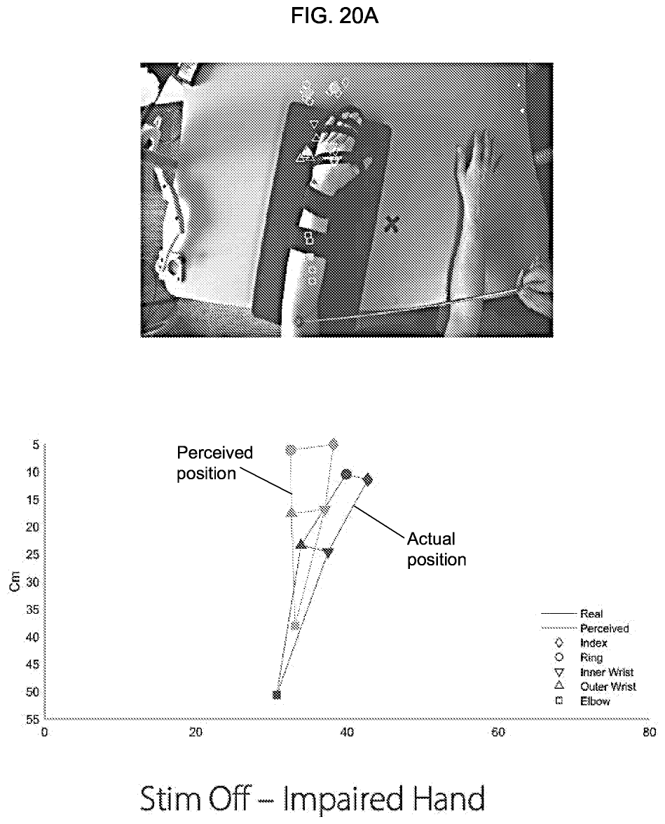

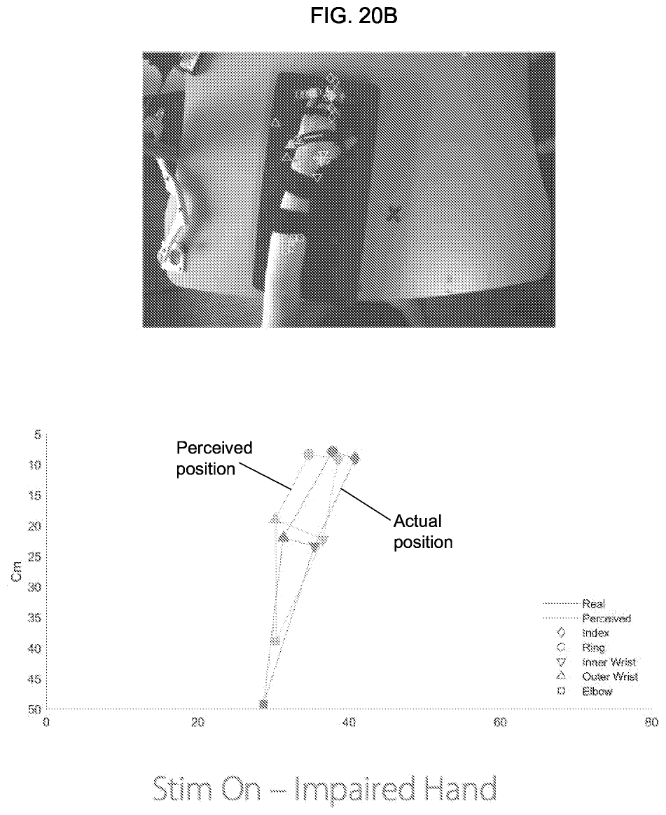

To determine whether SCS improves proprioception in the impaired arm, participants were assessed using a position matching proprioception task and a body representation proprioception task (FIGS. 19 and 20).

The position matching proprioception task evaluates the subject's ability to locate a precise location without vision, relying on their upper limb proprioception only. The participant was asked to reach two different targets. The kinematics of their movements were measured using a robotic platform. Trials were performed over three days, day 1 (FIG. 19A), day 2 (FIG. 19B), and day 3 (FIG. 19C). Consistently during the three days, application of SCS helped the participant to have a better space representation of their hand location in the working space. In other words, with stimulation ON, the subject demonstrated significantly better active movement proprioception than when stimulation was turned OFF. This was true across all three days indicated and all directions/targets tested.

The body representation proprioception task evaluates the participants' ability to recognize their upper limb part location in a static position (FIG. 20). The specific body locations the participants were asked to locate were the index finger, ring finger, inner wrist, outer wrist and elbow. While the participant was blindfolded, the impaired arm was occluded by concealing it under an opaque screen or box, so that the participant has no visual indication of the arm's position. Next, the participants were asked to focus on one of the body location listed above and the examiner would move a pointer above the obscured arm. The pointer was visible by the participant. Once the participant felt the pointer was over the arm location being tested, the pointer location was recorded and compared to the actual position of that body location. The results show that SCS stimulation improved both the ability of the subject to accurately perceive their body's position in space without the aid of visual feedback (body representation).

It will be apparent that the precise details of the methods described may be varied or modified without departing from the spirit of the disclosed subject matter. We claim all such modifications and variations that fall within the scope and spirit of the claims below.

1. A method for treating an impairment of a limb in a subject, comprising:
applying a therapeutically effective amount of an electrical stimulus to sensory neurons innervating the limb of the subject; and wherein:
the impairment is a motor impairment and/or a proprioception impairment due to neurological disorder or injury;
the electrical stimulus is applied with one or more electrodes controlled by a neurostimulator; and
application of the electrical stimulus reduces the motor impairment and/or proprioception impairment of the limb of the subject.

2. The method of claim 1, wherein:
the therapeutically effective amount of the electrical stimulus is applied to dorsal roots, dorsal rootlets, or dorsal root ganglia, of the sensory neurons innervating the limb of the subject; and
the one or more electrodes are implanted in the epidural space at the dorsolateral aspect of the spinal cord and proximate to the dorsal roots or dorsal rootlets of the sensory neurons innervating the limb of the subject, or the one or more electrodes are implanted proximate to dorsal root ganglia of sensory neurons innervating the limb of the subject.

3. The method of claim 1, wherein the sensory neurons are stimulated within the dorsal or ventral horn via epidural, subdural or intraspinal stimulation.

4. The method of claim 1, wherein the limb is an arm.

5. The method of claim 1, wherein the impairment is a motor impairment.

6. The method of claim 5, wherein the motor impairment is an impairment of voluntary movement of the limb.

7. The method of claim 5, wherein the motor impairment comprises reduced muscle control, reduced muscle function, reduced muscle strength, partial paralysis, uncontrollable muscle tone, reduced dexterity, spasticity, contractures, and/or abnormal flexor synergy or contractures.

8. The method of claim 5, wherein the subject retains at least some residual activity of corticospinal tract neurons innervating the impaired limb.

9. The method of claim 1, wherein the subject retains at least some residual movement of the impaired limb.

10. The method of claim 1, wherein the impairment is a proprioception impairment.

11. The method of claim 10, wherein the proprioception impairment comprises reduced ability to detect force generated by and/or applied to the limb.

12. The method of claim 10, wherein the proprioception impairment comprises reduced understanding of limb position and/or dynamics.

13. The method of claim 1, wherein the impairment is due to stroke.

14. The method of claim 1, wherein the impairment is due to cerebral palsy, traumatic brain injury, a neurodegenerative disease, a muscular dystrophy disease, amyotrophic lateral sclerosis, or Duchenne motor disorder.

15. The method of claim 1, wherein the impairment is not due to spinal cord injury.

16. The method of claim 1, wherein the one or more electrodes are implanted in the epidural space at the dorsolateral aspect of the spinal cord and proximate to the dorsal roots or dorsal rootlets of the sensory neurons innervating the limb of the subject.

17. The method of claim 1, wherein the one or more electrodes are implanted proximate to dorsal root ganglia of sensory neurons innervating the limb of the subject.

18. The method of claim 1, wherein the one or more electrodes are contained within an array of independently controllable electrodes on a percutaneous lead.

19. The method of claim 1, wherein the one or more electrodes are contained within an array of independently controllable electrodes on a paddle lead.

20. The method of claim 1, wherein the limb is an arm of the subject and the one or more electrodes are contained within one or more electrode arrays implanted at the dorsolateral aspect of the spinal cord and spanning the C3-T1 nerve roots.

21. The method of claim 1, wherein the limb is a leg of the subject and the one or more electrodes are contained within one or more electrode arrays implanted at the dorsolateral aspect of the spinal cord and spanning the T11-S1 nerve roots.

22. The method of claim 1, wherein the neurostimulator is an external or implanted pulse generator.

23. The method of claim 1, further comprising selecting the subject with the motor impairment and/or proprioception impairment for treatment.

24. The method of claim 1, wherein the electrical stimulus is applied at or below a motor threshold such that the electrical stimulus does not directly elicit movement of the impaired limb.

25. The method of claim 1, wherein the electrical stimulus is applied at or below a perceptual threshold such that the electrical stimulus does not directly elicit sensations in the impaired limb.

26. The method of claim 1, wherein the electrical stimulus is applied at or below a motor threshold such that the electrical stimulus does not directly elicit movement of the impaired limb, and above a perceptual threshold such that the electrical stimulus does elicit sensations in the impaired limb.

27. The method of claim 1, wherein the electrical stimulus does not induce paresthesia in the subject.

28. The method of claim 1, wherein the electrical stimulus increases the firing rate of motoneurons innervating the impaired limb of the subject, thereby reducing the impairment.

29. The method of claim 1, wherein the electrical stimulus increases the number of active motoneurons innervating the impaired limb of the subject, thereby reducing the impairment.

30. The method of claim 1, wherein the electrical stimulus activates sensory afferent cells of the spinal cord to increase the firing rate of intraspinal neural circuits and motoneurons innervating the impaired limb of the subject, thereby reducing the impairment.

31. The method of claim 1, wherein the electrical stimulus comprises:
electrical pulses having an amplitude of about 0.01 μA to about 50 mA, a pulse duration between about 40 μs and about 2 ms, and a frequency of about 1 Hz to about 1000 Hz.

32. The method of claim 1, wherein the electrical stimulus comprises electrical pulses having an amplitude of about 10 μA to about 20 mA, a pulse duration between about 40 μs and about 1 ms, and a frequency of about 10 Hz to about 300 Hz.

33. The method of claim 1, wherein the electrical stimulus comprises electrical pulses having an amplitude of about 10 μA to about 15 mA, a pulse duration between about 40 μs and about 500 μs, and a frequency of about 10 Hz to about 200 Hz.

34. The method of claim 1, wherein the electrical stimulus comprises a stimulation pattern of a series of 2 to 5 pulses separated by inter-pulse intervals of about 0.5 ms to about 10 ms and wherein the series is repeated at a frequency of about 10 Hz to about 500 Hz.

35. The method of claim 34, wherein the stimulation pattern is a series of 3 pulses separated by inter-pulse intervals of about 0.5 ms, wherein the series is repeated at a frequency of about 200 to about 500 Hz, and wherein the pulse duration is about 200 μs.

36. The method of claim 31, wherein the pulses are cathodic-first biphasic or monophasic charge balanced pulses.

37. The method of claim 1, wherein the neurostimulator is activated to apply the electrical stimulus in response to feedback from one or more sensors on the impaired limb.
